(19) United States
(12) Patent Application Publication
(10) Pub. No.: US 2025/0073014 A1
(43) Pub. Date: Mar. 6, 2025

(54) METHODS, DEVICES AND SYSTEMS FOR TREATING NEOINTIMAL GROWTH

(71) Applicants: James Biggins, Acton, MA (US); Rajesh Kumar, Nashua, NH (US)

(72) Inventors: James BIGGINS, Acton, MA (US); Rajesh Kumar, Nashua, NH (US)

(21) Appl. No.: 18/909,484

(22) Filed: Oct. 8, 2024

Related U.S. Application Data
(63) Continuation of application No. PCT/US2023/018490, filed on Apr. 13, 2023.
(60) Provisional application No. 63/330,949, filed on Apr. 14, 2022.

(51) Int. Cl. A61F 2/01

(52) U.S. Cl. CPC A61F 2/013

(57) ABSTRACT

The present invention relates generally to medical compositions, methods and devices/systems for treating vascular diseases. More particularly, the invention relates to medical methods, devices and kits for distributing drug to surrounding vascular tissue to treat neointimal growth. More specifically, the described invention is intended to overcome shortcomings of existing treatments for peripheral artery disease (PAD). Devices and methods of the present invention are specifically intended to treat patients with PAD involving the infrapopliteal (tibial) arteries, e.g., patients having a clinical indication for treatment below the knees (e.g., claudication and/or critical limb ischemia (CLI), and patients with complex disease states, for reducing one or more of morbidity; treatment complications in treated patient populations, a reduction in target lesion revascularization (TLR) rates; and a reduction in patients populations requiring any type of leg amputation.

### CROSS-REFERENCE TO RELATED APPLICATIONS

This application is a Continuation of, and claims priority to, PCT/US2023/018490 filed Apr. 13, 2023, now U.S. Pat. No. 11,771,710 issued Oct. 3, 2023; which claims benefit of U.S. Provisional Application Serial Nos. 63/330,949 filed Apr. 14, 2022, now expired, the contents of which are incorporated herein in their entirety.

### FIELD OF THE INVENTION

The present invention relates generally to medical compositions, methods and devices/systems for treating vascular diseases. More particularly, the invention relates to medical methods, devices and kits for distributing drug to surrounding vascular tissue to treat neointimal growth. More specifically, the described invention is intended to overcome shortcomings of existing treatments for peripheral artery disease (PAD). Devices and methods of the present invention are specifically intended to treat patients with PAD involving the infrapopliteal (tibial) arteries, e.g., patients having a clinical indication for treatment below the knees (e.g., claudication and/or critical limb ischemia (CLI), and patients with complex disease states, for reducing one or more of morbidity; treatment complications in treated patient populations, a reduction in target lesion revascularization (TLR) rates; and a reduction in patients populations requiring any type of leg amputation.

### BACKGROUND

Blockages can from in blood vessels under various disease conditions. In atherosclerosis, the narrowing of arteries in the body, particularly in the heart, legs, carotid, and renal anatomy, can lead to tissue ischemia from lack of blood flow. Mechanical revascularization methods, such as balloon angioplasty, atherectomy, stenting or surgical endarterectomy, may be used on blood vessels to open the vessel and to improve blood flow to downstream tissues. Unfortunately, mechanical revascularization can lead to an injury cascade that causes the blood vessel to stiffen and vessel walls to thicken with scar-like tissue, which can reduce blood flow and necessitate another revascularization procedure.

Moreover, while some drug delivery devices were shown to be in effective for treating lesions above the knee, these devices were not able to effectively treat small vessels, e.g. below the knee. Importantly, current devices have set lengths so the same device is not able to treat variable lesion lengths without resorting to having to use using multiple types of devices or different lengths of devices.

Therefore, there is a need for treatments of blood vessels for reducing vessel stiffening and thickening following mechanical revascularization to maintain or improve the patency of the blood vessels.

### SUMMARY OF THE INVENTION

The present invention relates generally to medical compositions, methods and devices/systems for treating vascular diseases. More particularly, the invention relates to medical methods, devices and kits for distributing drug to surrounding vascular tissue to treat neointimal growth. More specifically, the described invention is intended to overcome shortcomings of existing treatments for peripheral artery disease (PAD). Devices and methods of the present invention are specifically intended to treat patients with PAD involving the infrapopliteal (tibial) arteries, e.g., patients having a clinical indication for treatment below the knees (e.g., claudication and/or critical limb ischemia (CLI), and patients with complex disease states, for reducing one or more of morbidity; treatment complications in treated patient populations, a reduction in target lesion revascularization (TLR) rates; and a reduction in patients populations requiring any type of leg amputation.

Blockages can from in the blood vessels under various disease conditions. Atherosclerosis which causes a narrowing or stenosis of arteries in the body, particularly in heart, legs, carotid and renal anatomy, can lead to tissue ischemia from lack of blood flow. Atherosclerosis in the coronary arteries can cause myocardial infractions, commonly referred to as heart attack, which can be immediately fatal or even if survived can cause damage to the heart which can incapacitate the patient. Other coronary artery diseases include congestive heart failure, vulnerable or unstable plaque, in cardiac arrhythmias which cause death and incapacitation. In addition, peripheral artery disease or PAD, where the arteries in the peripheral tissue narrow, most commonly affecting the legs, renal, and carotid arteries. Blood clots and thrombosis, e.g., debris, in the peripheral vasculature may flow to other parts of the body leading to tissue and organ necrosis. Some patients with PAD experience critical limb ischemia which can result in ulcers and can require partial or full leg amputation, in the worst cases. PAD in a renal artery can cause renovascular hypertension while clots in the carotid arteries can embolize and travel in the brain, potentially causing ischemic stroke.

Current methods of vessel treatment can fail because they: create mechanical damage to the tissue; require high doses of the active ingredient to be effective, thus increasing risk of side effects; do not distribute drug evenly on the surface of the target tissue; create fragments of different types resulting from deployment of the device, including but not limited to flaking off of drug coating from the drug coated device, and debris release (ruptured cellular components and/or basement membrane from the manipulated tissue, use a solid drug component that is not water soluble which gets pushed into the tissue wall in order to treat the target lesion; they coat devices and this commonly requires an excipient to attach the drug to the device and the drug coating is inconsistent, e.g., uneven, on the devices, etc. Although some drug delivery devices were shown to be in effective for treating some lesions, such devices were not able to effectively treat small vessels.

The invention described herein, is intended to address the aforementioned shortcomings of existing treatments. Embodiments include devices specifically intended to treat patients with PAD involving the infrapopliteal (tibial) arteries including patients having a clinical indication for treatment, e.g., Claudication, which literally means “to limp,” is one of the symptoms of lower extremity peripheral artery disease (PAD), critical limb ischemia (CLI) referring to a severe blockage in the arteries of the lower extremities which markedly reduces blood-flow, and patients with complex disease states, e.g., PAD with coronary artery disease (CAD), PAD with Diabetes mellitus. Benefits of using devices, systems and methods described herein are contemplated to include but not limited to, a reduced morbidity and complications in treated patient populations, e.g., showing superior patency at 6 months with a reduction in target lesion revascularization (TLR) rates; a reduction in patients populations requiring any type of leg amputation, etc. “Target lesion revascularization” refers to any procedure performed to restore luminal patency after there has been late luminal loss attributable to in-stent restenosis (ISR).

Certain embodiments described below have a number of advantages including a) eliminating the balloon decoration with drug, b) easy formulation, c) no need to prepare or synthesize drug carrier, d) high output, or drug bioavailability, e) fast delivery, and f) easy handling. In addition, the approaches describe below minimize i) particle loss, ii) mechanical stress put onto the vessel during therapy, and iii) exposure to drug and total drug needed, reducing side effects.

In some embodiments, the present invention contemplates stopping blood flow in order to treat the target tissue. Because the vasculature is a closed system, the blood flow maybe stopped with a distal (downstream) or proximal (upstream) flow occlusion (or both). Thus, in one embodiment, the present invention contemplates a method of treating a target tissue exhibiting neointimal growth in a vessel in a subject, comprising: a) occluding said vessel in said subject with a device, at least a portion of said device positioned downstream past the target tissue exhibiting neointimal growth, said portion stopping blood flow at the distal end of said vessel; b) introducing a treatment solution comprising a growth inhibitor or other drug so that it contacts said target tissue for a period of time (e.g. 1 to 100 minutes, more typically 2 to 30 minutes); and c) removing said device from said vessel, thereby returning blood flow at the distal end of said vessel. In one embodiment, the growth inhibitor is Sirolimus. In one embodiment, Sirolimus is introduced in or on a nanoparticle. In one embodiment, Sirolimus is incorporated during nanoparticle production. In one embodiment, Sirolimus is encapsulated in a plurality of nanoparticles. In one embodiment, Sirolimus is encapsulated in gelatin nanoparticles (GNPs).

In another embodiment, the present invention contemplates a method of treating a target tissue exhibiting neointimal growth in a vessel in a subject, comprising: a) occluding said vessel in said subject with a device, at least a portion of said device positioned upstream of the target tissue exhibiting neointimal growth, said portion stopping blood flow at the proximal end of said vessel; b) introducing a treatment solution comprising a growth inhibitor so that it contacts said target tissue for a period of time (e.g. 1 to 100 minutes, more typically 2 to 30 minutes); and c) removing said device from said vessel, thereby returning blood flow at the distal end of said vessel. It is not intended that the present invention be limited to a specific growth inhibitor or treatment solution for these embodiments. In one embodiment, the growth inhibitor is paclitaxel. In another embodiment, the growth inhibitor is Sirolimus (also known as rapamycin). In one embodiment, rapamycin is in a treatment solution comprising dimethylsulfoxide (DMSO). In one embodiment, rapamycin is introduced in or on a nanoparticle. In one embodiment, rapamycin is incorporated during nanoparticle production. In one embodiment, rapamycin is encapsulated in a plurality of nanoparticles. In one embodiment, rapamycin is encapsulated in gelatin nanoparticles (GNPs). In a preferred embodiment, the treatment solution at step b) soaks the target tissue such that the treatment solution (or portion thereof) penetrates the tissue to permit the drug to enter the vessels, e.g. vessel wall. In one embodiment, said device is a hypotube comprising a channel. In one embodiment, said hypotube further comprising one or more openings, e.g., holes such as microholes. In a preferred embodiment, said treatment solution is introduced through said channel of said hypotube and is release through said openings, e.g., microholes, to the target tissue. In one embodiment, the hypotube is covered at least in part by a restriction catheter. In one embodiment, the present invention further contemplates moving the restriction catheter to restrict or increase the amount of therapeutic and location of the therapeutic being eluted by the hypotube, thereby allowing for the variable length of the vessels and lesions therein. In other words, in one embodiment, the present invention contemplates telescoping a hollow restriction catheter over a hypotube comprising pores allows for variable length expulsion of drug from open holes that are not covered by the surrounding restriction catheter, in order to provide treatment to different lesion lengths using one device/system. In one embodiment, said device is a hypotube carrying a filter. In one embodiment, said filter is deployed like an umbrella at the distal end to stop blood flow at said distal end. In one embodiment, said filter is deployed like an umbrella at the proximal end to stop blood flow at said proximal end.

Whether deployed at the distal or proximal ends, the filter can also function to stop particles, e.g., potential emboli, from escaping the target site. In one embodiment, said vessel contains a partial or complete blockage at or near said target tissue. In one embodiment, the method further comprises prior to step b) of any of the embodiments described above, performing a procedure to remove or reduce said blockage. It is not intended that the present invention be limited by the nature of the procedure to remove or reduce said blockage. In one embodiment, said procedure is a chemical procedure that dissolves at least a portion of said blockage, e.g. calcification removal using a chemical method (discussed more below). In one embodiment, said chemical procedure comprises delivering a chemical through said channel of said hypotube for a period of time (e.g., 1 to 100 minutes, more typically 2 to 30 minutes), said hypotube further comprising holes, e.g., microholes, such that the blockage is irrigated by the chemical coming through the microholes. It is not intended to limit the chemical, nonlimiting examples include amine and alcohol-based solvents, chelating agents, Papain enzyme, etc. Nonlimiting examples of amine-based compounds include but are not limited to urazole, glutamate, solvents, e.g., ethanol with octanol or octanediol, etc. Nonlimiting examples of chelating agents include but are not limited to disodium ethylene diamine tetra acetic acid (EDTA), diethylene triamine penta acetic acid (DTPA) and sodium thiosulfate (STS), etc.

In another embodiment, said procedure is a surgical procedure. In one embodiment, said surgical procedure is angioplasty. In another embodiment, said surgical procedure is an atherectomy. In one embodiment, said atherectomy is selected from the group consisting of rotational, transluminal, and directional atherectomy. In one embodiment, the method (of any of the embodiments discussed above) further comprises, prior to step b), but after performing said procedure to remove or reduce said blockage, removing particles generated by said procedure. The particles can be removed in a variety of ways, and even with a combination of ways. In one embodiment, said particles are potential emboli and they are removed with an evacuation catheter. In one embodiment, the particles are blocked by said filter (discussed above) and removed when the filter is removed. In one embodiment, particles are removed by both an evacuation catheter and said filter. It is not intended that the present invention be limited to the nature or size of the vessel treated with any of the embodiments discussed above. The present invention contemplates treating vessels (whether arteries or veins) in the arms, legs and torso of animals and humans. However, in a preferred embodiment, treatment is contemplated for the vessels (arteries and veins) shown in FIG. 28, and in particular vessels at or below the knee area.

For example, in any of the embodiments described above, treatment may be performed on a popliteal artery or vein. Similarly, in any of the embodiments described above, treatment may be performed on a tibial artery or vein. Typically, for any of the embodiments described above, said target tissue exhibiting neointimal growth is in a vessel below the knee of said subject. In one embodiment, the portion of said device positioned downstream past the target tissue exhibiting neointimal growth is a distal balloon that is inflated to stop blood flow at the distal end of said vessel. In one embodiment, the portion of said device positioned upstream of the target tissue exhibiting neointimal growth is a proximal balloon that is inflated to stop blood flow at the proximal end of said vessel. In other embodiments with the distal balloon, said device also has an associated proximal balloon which is positioned upstream to block proximal blood flow.

As noted above, in some embodiments, the present invention contemplates stopping blood flow in order to treat the target tissue. Because the vasculature is a closed system, the blood flow maybe stopped with a distal (downstream) or proximal (upstream) flow occlusion. Thus, in one embodiment, the present invention contemplates a method of treating a target tissue exhibiting neointimal growth in a vessel in a subject, comprising: a) occluding said vessel in said subject with a device, at least a portion of said device positioned either upstream of the target tissue or downstream past the target tissue exhibiting neointimal growth, said portion stopping blood flow either at the proximal or distal end of said vessel; b) introducing a treatment solution comprising rapamycin and dimethylsulfoxide (DMSO) so that it contacts said target tissue for a period of time (e.g. 1 to 100 minutes, more typically 2 to 30 minutes); and c) removing said device from said vessel, thereby returning blood flow at the distal end of said vessel. In a preferred embodiment, the treatment solution at step b) soaks the target tissue such that the treatment solution (or portion thereof) penetrates the tissue to permit the drug to enter the vessels, e.g., vessel wall. In one embodiment, rapamycin is introduced in or on a nanoparticle. In one embodiment, rapamycin is incorporated during nanoparticle production. In one embodiment, rapamycin is encapsulated in a plurality of nanoparticles. In one embodiment, rapamycin is encapsulated in gelatin nanoparticles (GNPs). In one embodiment, said device is a hypotube comprising a channel. In one embodiment, said hypotube further comprising one or more openings, e.g., holes such as microholes. In a preferred embodiment, said treatment solution is introduced through said channel of said hypotube and is release through said openings, e.g., microholes, to the target tissue. In one embodiment, the hypotube is covered at least in part by a restriction catheter. In one embodiment, the present invention further contemplates moving the restriction catheter to restrict or increase the amount of therapeutic and location of the therapeutic being eluted by the hypotube, thereby allowing for the variable length of the vessels and lesions therein. In other words, telescoping a hollow restriction catheter over a hypotube comprising pores allows for variable length expulsion of drug from open holes that are not covered by the surrounding catheter, in order to provide treatment to different lesion lengths using one device/system. In one embodiment, said device is a hypotube carrying a filter. In one embodiment, said filter is deployed like an umbrella at the distal end to stop blood flow at said distal end. In one embodiment, said filter is deployed like an umbrella at the proximal end to stop blood flow at said proximal end. Whether deployed at the distal or proximal ends, the filter can also function to stop particles, e.g. potential emboli, from escaping the target site. In one embodiment, said vessel contains a partial or complete blockage at or near said target tissue. In one embodiment, the method further comprises prior to step b) of any of the embodiments described above, performing a procedure to remove or reduce said blockage. It is not intended that the present invention be limited by the nature of the procedure to remove or reduce said blockage. In one embodiment, said procedure is a chemical procedure that dissolves at least a portion of said blockage, e.g. calcification removal using a chemical method. In one embodiment, said chemical procedure comprises delivering a chemical through said channel of said hypotube for a period of time (e.g. 1 to 100 minutes, more typically 2 to 30 minutes), said hypotube further comprising holes, e.g. microholes, such that the blockage is irrigated by the chemical coming through the microholes. In another embodiment, said procedure is a surgical procedure. In one embodiment, said surgical procedure is angioplasty. In another embodiment, said surgical procedure is an atherectomy. In one embodiment, said atherectomy is selected from the group consisting of rotational, transluminal, and directional atherectomy. In one embodiment, the method (of any of the embodiments discussed above) further comprises, prior to step b), but after performing said procedure to remove or reduce said blockage, removing particles generated by said procedure. The particles can be removed in a variety of ways, and even with a combination of ways. In one embodiment, said particles are potential emboli and they are removed with an evacuation catheter. In one embodiment, the particles are blocked by said filter (discussed above) and removed when the filter is removed. In one embodiment, particles are removed by both an evacuation catheter and said filter. It is not intended that the present invention be limited to the nature or size of the vessel treated with any of the embodiments discussed above. The present invention contemplates treating vessels (whether arteries or veins) in the arms, legs and torso of animals and humans. However, in a preferred embodiment, treatment is contemplated for the vessels (arteries and veins) shown in FIG. 28, and in particular vessels at or below the knee area.

For example, in any of the embodiments described above, treatment may be performed on a popliteal artery or vein. Similarly, in any of the embodiments described above, treatment may be performed on a tibial artery or vein. Typically, for any of the embodiments described above, said target tissue exhibiting neointimal growth is in a vessel below the knee of said subject. In one embodiment, the portion of said device positioned downstream past the target tissue exhibiting neointimal growth is a distal balloon that is inflated to stop blood flow at the distal end of said vessel. In one embodiment, the portion of said device positioned upstream of the target tissue exhibiting neointimal growth is a proximal balloon that is inflated to stop blood flow at the proximal end of said vessel. In other embodiments with the distal balloon, said device also has an associated proximal balloon which is positioned upstream to block proximal blood flow.

Again, as noted above, in some embodiments, the present invention contemplates stopping blood flow in order to treat the target tissue. Because the vasculature is a closed system, the blood flow maybe stopped with a distal (downstream) or proximal (upstream) flow occlusion, e.g. a distal or proximal balloon (or both) that, when inflated, stops blood flow. Thus, in one embodiment, the present invention contemplates a method of treating a target tissue exhibiting neointimal growth in a vessel in a subject, comprising: a) providing a device comprising a hypotube comprising a channel and one or more openings or holes, such as microholes, said hypotube having an associated distal (or proximal) balloon; b) introducing said device into said vessel in said subject, at least a portion of said device positioned downstream past the target tissue exhibiting neointimal growth, said portion comprising the associated distal balloon (or, in the alternative, positioning a proximal balloon upstream of the target tissue); c) inflating said associated distal (or proximal) balloon thereby stopping blood flow at the distal end of said vessel; d) introducing a treatment solution comprising a growth inhibitor into said hypotube and out of said microholes so that said treatment solution contacts said target tissue for a period of time (e.g. 1 to 100 minutes, more typically 2 to 30 minutes); and e) deflating said associated distal (or proximal) balloon and removing said device from said vessel, thereby returning blood flow in said vessel. It is not intended that the present invention be limited to a specific growth inhibitor or treatment solution for these embodiments. In one embodiment, the growth inhibitor is paclitaxel. In another embodiment, the growth inhibitor is rapamycin. In one embodiment, rapamycin is introduced in or on a nanoparticle. In one embodiment, rapamycin is incorporated during nanoparticle production. In one embodiment, rapamycin is encapsulated in a plurality of nanoparticles. In one embodiment, rapamycin is encapsulated in gelatin nanoparticles (GNPs). In one embodiment, said rapamycin is in a treatment solution comprising dimethylsulfoxide (DMSO). In a preferred embodiment, the treatment solution at step b) soaks the target tissue such that the treatment solution (or portion thereof) penetrates the tissue to permit the drug to enter the vessels, e.g. vessel wall. In one embodiment, the hypotube is covered at least in part by a restriction catheter. In one embodiment, the present invention further contemplates moving the restriction catheter to restrict or increase the amount of therapeutic and location of the therapeutic being eluted by the hypotube, thereby allowing for the variable length of the vessels and lesions therein. In other words, telescoping a hollow restriction catheter over a hypotube comprising pores allows for variable length expulsion of drug from open holes that are not covered by the surrounding catheter, in order to provide treatment to different lesion lengths using one device/system. In one embodiment, said hypotube further comprises a filter. Whether deployed at the distal or proximal ends, the filter can also function to stop particles, e.g., potential emboli, from escaping the target site. In one embodiment, said vessel contains a partial or complete blockage at or near said target tissue. In one embodiment, said vessel contains a partial or complete blockage at or near said target tissue. In one embodiment, the method further comprises prior to step d) of the embodiments described above, performing a procedure to remove or reduce said blockage. It is not intended that the present invention be limited by the nature of the procedure to remove or reduce said blockage. In one embodiment, said procedure is a chemical procedure that dissolves at least a portion of said blockage, e.g. calcification removal using a chemical method. In one embodiment, said chemical procedure comprises delivering a chemical through said channel of said hypotube and through said one or more openings in said hypotube for a period of time (e.g., 1 to 100 minutes, more typically 2 to 30 minutes), said openings comprising holes, e.g., microholes, such that the blockage is irrigated by the chemical coming through the opening(s). In another embodiment, said procedure is a surgical procedure. In one embodiment, said surgical procedure is angioplasty. In another embodiment, said surgical procedure is an atherectomy. In one embodiment, said atherectomy is selected from the group consisting of rotational, transluminal, and directional atherectomy. In one embodiment, the method (of any of the embodiments discussed above) further comprises, prior to step d), but after performing said procedure to remove or reduce said blockage, removing particles generated by said procedure. The particles can be removed in a variety of ways, and even with a combination of ways. In one embodiment, said particles are potential emboli and they are removed with an evacuation catheter. In one embodiment, the particles are blocked by said filter (discussed above) and removed when the filter is removed. In one embodiment, particles are removed by both an evacuation catheter and said filter. It is not intended that the present invention be limited to the nature or size of the vessel treated with any of the embodiments discussed above. The present invention contemplates treating vessels (whether arteries or veins) in the arms, legs and torso of animals and humans. It is not intended that the present invention be limited to the means by which the balloon is deflated. In one embodiment, the deflation is done by aspirating the saline solution present in the inflated balloons.

However, in a preferred embodiment, treatment is contemplated for the vessels (arteries and veins) shown in FIG. 28, and in particular vessels at or below the knee area. For example, in any of the embodiments described above, treatment may be performed on a popliteal artery or vein. Similarly, in any of the embodiments described above, treatment may be performed on a tibial artery or vein. Typically, for any of the embodiments described above, said target tissue exhibiting neointimal growth is in a vessel below the knee of said subject. In one embodiment, the portion of said device positioned downstream past the target tissue exhibiting neointimal growth is a distal balloon that is inflated to stop blood flow at the distal end of said vessel. In one embodiment, the portion of said device positioned upstream of the target tissue exhibiting neointimal growth is a proximal balloon that is inflated to stop blood flow at the proximal end of said vessel. In other embodiments with the distal balloon, said device also has an associated proximal balloon which is positioned upstream to block proximal blood flow.

In one embodiment, the present invention contemplates a system or kit, comprising i) a hypotube comprising a channel and one or more openings such as holes, e.g. microholes, said hypotube having an associated distal balloon and/or an associated proximal balloon, ii) an evacuation catheter with an open lumen to the inner section between the distal and proximal balloons; and iii) a restriction catheter to allow for variable length to accommodate different lesion lengths. The restriction catheter, in one embodiment, is configured like a covering or sheet that is over the smaller hypotube delivering the therapeutic. The restriction catheter can be moved distally approximately in relation to the distal balloon to restrict the amount of therapeutic and location of the therapeutic being eluted by the hypotube, thereby allowing for the variable length of the vessels and lesions therein. As an example, if the target lesion is only 20 mm in length the restriction catheter can modify the length of hypotube that is releasing the therapeutic. If the lesion is much longer, e.g. 150 mm, then restriction catheter can be pulled back so that more of the holes in the hypotube are exposed, thereby releasing the therapeutic agent (in the treatment solution) over a longer length.

In another embodiment, the present invention contemplates a device comprising a combination of a wire and a hypotube attached to a balloon or other mechanical occlusion device to the distal end. A small hypotube surrounding a wire would be used to deliver the drug through a plurality of holes (pores), hypotube, i.e. perforated. As the drug is flowed through the hypotube it would elude into the vasculature including the blood. An additional feature with this device is a means to change the length or distance that the drug is being introduced into the vasculature, therefore allowing for treating lesions of different lengths using the same device. In a further embodiment, an additional tube, e.g., restriction catheter, is provided for surrounding a portion of the perforated hypotube for covering over some of the holes in the hypotube that would block off flow of the treatment solution (such as DMSO+rapamycin) to avoid treating that section of vessel, and in turn target treat a specific length of vessel. FIG. 5 is illustrative of one embodiment of this type.

In one embodiment, the balloon is moved proximately to prevent blood flow. In some embodiments, a filter is placed distally from the balloon in order to prevent emboli. FIG. 6 is illustrative of one embodiment of this type.

In one embodiment, a balloon is located proximally to prevent blood flow while a distal hypotube with perforated sides allows drug to be irrigated into the vessels. FIG. 7 is illustrative of one embodiment of this type.

In one embodiment, a device and/or system further includes one or more of a hypotube, wire, balloon, with a microcatheter as an occlusive mechanism on the distal end of a treatment area, with a balloon occluding the proximal end of the treatment area. In a further embodiment, a catheter pushes a treatment formulation into the vessel area targeted for treatment. In some embodiments, a radiopaque, e.g., iodine in fluid may be added to the DMSO+rapamycin formulation, and pieces of radiopaque material attached to wires, catheters, inside balloons, etc., providing a means for a clinician to clearly identify the area of the vessel being treated during the procedure using x-rays or other radiation emitting equipment. Radiopaque materials include but are not limited to small molecular weight salts or compounds or nanoparticles containing iodine, barium, tantalum, bismuth, or gold. FIG. 8 is illustrative of one embodiment of this type. Additionally included in a device embodiment is an evacuation catheter for the evacuation of thrombi or emboli after the treatment procedure (whether chemical or surgical) is completed. The catheter might also be used in place of a hypotube for deploying fluids, e.g., drug solution, and extracting and/or evacuating remaining blood within the treatment area or remnants of the treatment, e.g., emboli, debris, etc. The distance of the catheter from the balloon would be adjusted to accommodate for treating lesions of different lengths as described herein.

In one embodiment, the device further comprises (in addition to the features illustrated by FIGS. 3-7) an additional feature of a balloon surrounding a hypotube or catheter for methods ensuring evacuation of blood, drug delivery to a target vesicular wall area and removal of drug solution after treatment. Exemplary FIG. 9 is illustrative of one embodiment of this type.

In one embodiment, a device comprises three balloons along a wire, wherein the proximal balloon surrounds a tube, for use in a treatment method by inflating the center balloon to evacuate blood in an area designated for treatment, followed by inflating the two end balloons to block off the vessel treatment area, followed by deflating the center balloon while backfilling the vacant space with a fluid introduced through the tube, e.g., drug solution. In other words, First stage: step 1: inflate the center balloon to evacuate the area of any blood, step 2 inflate the distal and proximal balloons, a second stage would include deflating the center balloon and introducing drug formulation within the center areas (hatched), after the blood has been evacuated. FIG. 10 is illustrative of one embodiment of this type. It is not intended that the present invention be limited to the means by which the balloon(s) is/are deflated. In one embodiment, the deflation is done by aspirating the saline solution present in the inflated balloons.

In one embodiment, a device comprises a hollow empty space (hard boundaries) created between two balloons, located longitudinally on top of one another with a space longitudinally located in between these 2 balloons where the space allows blood flow in between these balloons. Each balloon has multiple pores, e.g., micropores, along their luminal sides, and are filled with a drug solution, e.g., a Sirolimus solution, in either 100% DMSO or a mixture of DMSO and saline with little leakage through pores during travel through vessels to the treatment area. During deployment where the device is held in place, balloons are simultaneously inflated, just enough that the sides with the pores contact the vessel walls securely followed by inflation of the balloons to create pressure, e.g., squeezing, in order to allow the drug solution to pour out of the balloons through the pores and onto the vessel wall tissue. Absorption of DMSO into the vessel walls is contemplated as quick with a high delivery efficiency. The space between the two balloons is enough to allow continual blood throughflow which should ensure that the drug solution does not contact the blood stream and hence no loss of drug due to the flush of blood stream through this area as compared to losses using a conventional Drug Coated Balloon (DCB). FIG. 11 is illustrative of one embodiment of this type.

In one embodiment, a delivery system is provided comprising a medical grade flexible hollow plastic tube perforated with micropores around the outside of the tube, having end enclosures impermeable to fluids, wherein when filled with a fluid, e.g., a DMSO drug formulation, the fluid does not move through the micropores. Upon deployment in a treatment area, micropores would be adjacent to vessel walls and not the main stream of blood. Such plastic hollow tubes have outside boundaries of very thin and flexible plastic or textile (impermeable to fluid) which upon deployment in a vessel, responds to shear pressure generated by blood flow through the center area wherein the blood's hydrostatic pressure pushes the outer area of tubing against the vessel wall while pushing the drug formulation through the micropores and onto the vessel wall tissue. In other words, after the slowly moving blood through the narrow opening during deployment, when the tubes are held in position, blood then enters into the enlarging opening with greater force and higher volume as the tube is pushed towards the vessel walls for delivery an active agent, then tubes begin to deflate due to loss of fluid introduced into surrounding vessel wall tissue. FIG. 12 is illustrative of one embodiment of this type.

In one embodiment, a method of treatment using a device as described herein for treating thickened vessel wall areas, e.g., atherosclerotic plaques, PAD, etc., comprises preparation of the plaque covered vessel wall intended for treatment in the form of restoring blood flow through the plaque either using angioplasty or atherectomy. Thus, in one preferred embodiment, a method of treatment incorporates an angioplasty or atherectomy procedure prior to treatment with a device described herein, for combining within a complete procedure into a method of using the device. In one embodiment, atherectomy procedures include but are not limited to, rotational, transluminal, and directional atherectomy. In one embodiment, atherectomy procedures are used for below the knee applications.

There are at least two ways to stop the movement of emboli into the extremities that arise distal of devices used for treatment. One is filtration of the blood, in other words using a device having an attached filter or using with a device a distal protection filter. One is evacuating the disrupted tissue using a catheter so as not to allow distal movement of the emboli downstream of blood flow. Another is by using a method for treating arthrosclerosis comprising a chemical method of dissolving or removing the atherosclerotic plaque combined with delivering a treatment to the vessel wall in contact with plaque by using a device as described herein. In one embodiment, a device described herein having microholes or pores in the outer surface provides a chemical profusion through the profusion through the hypotube (infusion through the center of the hydrotube and irrigating into the vessel thought the microholes (micropores) depicted in FIG. 12. Infusion would happen for a period long enough for the chemical formulation to dissolve the target lesion but short enough so as to not cause clinical issues associated with stopping blood flow (e.g. 1 to 100 minutes, more typically 2 to 30 minutes). Once sufficient erosion of the target lesion is achieved the fragmented lesion would be evacuated through the evacuation catheter also depicted in FIG. 12. Once the lesion is removed, flow is sufficiently restored, and particulate/emboli is evacuated the drug solution could then be infused into the target area. Aspects of this embodiment include a hypotube to keep a low profile of the delivery system also enable the crossing through difficult lesions also known as “pushability.” The evacuation catheter with open lumen to the inner section between the two balloons so that blood and emboli can be removed, and its associated proximal balloon for stopping flow. Another aspect of this embodiment is a restriction catheter telescoping (sliding) over a hypotube comprising pores to allows for variable length expulsion of drug from open holes, not covered by the surrounding catheter, in order to provide treatment to different lesion lengths using one device/system. FIG. 13 is illustrative of one embodiment of this type.

A better understanding of the features and advantages of the present disclosure are also described herein in other subsections, including in the detailed description, that sets forth illustrative embodiments of devices, systems, compositions and methods of treatment, and in accompanying drawings. Further, compositions and methods described herein, including treating patients using DMSO as part of a drug formulation, may also include commercial devices whose exemplary components are described herein and shown in Figures.

In one embodiment, the present invention contemplates a delivery system comprising a delivery device comprising first, second and third ports, said first port configured to supply fluid through a first channel for inflating an angioplasty balloon, said second port configured to supply fluid through a second channel for inflating a distal balloon to block blood flow, said third port configured to supply fluid through a third channel in said hypotube, for supplying a treatment solution into a vessel. In one embodiment, said treatment solution comprises rapamycin encapsulated into a nano carrier. In one embodiment, said channels are positioned within a hypotube. FIG. 29 is illustrative of one embodiment of this type.

In another embodiment, the present invention contemplates a delivery system comprising a delivery device comprising first, second and third ports, said first port configured to supply fluid through a first channel in a hypotube, said fluid for inflating an angioplasty balloon, said second port configured to supply fluid through a second channel in said hypotube, said fluid for inflating a distal balloon to block blood flow, said third port configured to supply fluid through a third channel in said hypotube, for supplying a treatment solution into a vessel. In one embodiment, said treatment solution comprises rapamycin encapsulated into a nano carrier.

The following embodiments are merely illustrative. One or more embodiments may be combined for providing a device, steps in a treatment, and for overcoming or solving issues with using current devices and treatments, including but not limited to PAD and other vesicular medical treatments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an exemplary embodiment comprising a wash (or flush) technique using a device delivering a treatment solution, such as DMSO+a drug formulation, and more preferably a DMSO+rapamycin formulation, directly to the wall of the vessel (vessel wall 20) while preferably preventing or reducing mixing formulation with blood. After balloons 10 are deployed, arrows depict inflow of fluid through a hollow tube (e.g., hypotube or catheter 30) then flowing through one of the balloons 10 for introducing fluids, such as a drug formulation, and (optionally) washing fluids, etc., into the isolated space between the balloons. Conversely, arrows also show fluid flow for removal, such as blood, drug formulation after treatment, wash fluids, exiting the isolated space, etc.

FIG. 2 shows an exemplary embodiment for maximizing the amount of space 30 a balloon 10 takes up within the inside diameter of the vessel (e.g., as determined by a wall of the balloon 15). This would allow the DMSO plus reformation formulation to be present against the wall of the target vessel (vessel wall 20) thus minimizing the exposure of the formulation to the bloodstream. In certain embodiments, any emboli created from the inflation and deployment of the balloon as relates to the target tissue, are addressed. In addition, steps are taken to address the length of the vessel that receives an active ingredient, e.g., drug.

FIG. 3 shows an exemplary embodiment for delivering the DMSO+rapamycin formulation directly to the wall of the vessel (e.g., vessel wall 50) for reducing or preferably preventing mixing of the treatment solution (including but not limited to a DMSO+drug formulation) with blood. In one embodiment, a step 1 is evacuating blood by using a balloon in balloon device system (including one balloon 10 inside of another balloon 40), wherein the inner balloon 10 may be filled, for example, by injecting saline through a hypotube 80 (along a wire 30) into the inner balloon 10 at 25, where the inner balloon 10 has a wall 15 that does not have pores/holes. As the inner balloon 10 is inflating, it causes the outer balloon 40 to inflate (or a fluid is also pumped into balloon 40) resulting in the expanding wall of the inner balloon 15 pressing the wall 45 of the outer balloon 40 against the vessel wall 55. The outer balloon has pores 60 in its outer wall 45, at least in one area of outer balloon 40, for dispensing a treatment solution through hypotube 70 in an area of target tissue 55 in the vessel wall 50. Thus, allowing release of a treatment (drug) as its injected through hypotube 70 whose opening 75 is located in between the outer wall of the inner balloon and the wall of the outer balloon having pores/holes 60 for release of treatment directly onto vessel walls. In certain embodiments, disruption of the tissue and potential creation of emboli during deployment, are addressed. In addition, steps are taken to limit the length of vessel to be treated. In further embodiments, smaller diameters of vessels less than 4 millimeters would have a delivery system that would sufficiently deliver an active ingredient formulation. Loss of the formulation and generation of emboli when removing the device post treatment, both issues with most balloon-based delivery systems, are addressed.

FIG. 4 shows an exemplary embodiment for a low-profile system comprising a small balloon 10 attached to the distal or proximal end of a guide wire 30 and/or hypotube 20 to stop or inhibit blood movement within the vessel 40 (could be a balloon or mechanical occlusion feature). A filter 60 may be attached to the distal end. In this embodiment, blood flow is inhibited or stopped (e.g., step 1) until after releasing the treatment solution (including but not limited to a DMSO+rapamycin formulation) into the bloodstream for a specified time period. As the treatment solution mixes with the blood upstream from the occlusion feature, vessels may be treated in the stopped blood, for one example, a time period of between 2 to 30 minutes (or more) while the drug formulation is readily absorbed by the vessel wall target area 45, such as vessel walls 45. After treatment, balloon 10 is deflated for removal of hypotube 20/wire 30 (e.g., step 2). This type of embodiment has several distinct advantages including 1) a low-profile system through use of a small hypotube 20 as a wire 30 for delivering the balloon/delivery system to the target site and 2) essentially has the ability to deliver a delivery system to the target vasculature regardless of the size or length of the vessel merely by changing where the balloon is inflated along the hypotube 20 wire 30. The ability to treat very small vessels with minimal disruption to the vasculature and therefore minimizing any potential emboli created during the procedure itself is one advantage of using this method.

Additionally, in this embodiment, is the presence of a filter 60 on the distal end of wire 30 could be used for multiple beneficial results including preventing any emboli created during deployment of the balloon 10 or created from the DMSO drug combination from entering the blood stream during or after treatment while the device is being removed. After deflation of the balloon or removal of another type of occlusion feature.

FIG. 5 shows an exemplary embodiment comprising a wire 30 with hypotube 40 comprising pores 60, combination to attach a balloon 10 or mechanical occlusion device to the distal end. There's a small hypotube 40 that would be used to deliver the drug through many holes 60 (pores) in the hypotube 40 as the drug is flowed through the hypotube it would elude into the vasculature including the blood (vessel walls) 20. The additional feature to highlight here is a way to change (adjust) the length or distance that the drug is being introduced into the vasculature therefore accounting for variation in lesion length, e.g., adjustable sliding—telescoping catheter 50, tube covering, and the like). In this embodiment, this could be accomplished in many ways including a tube 50, e.g., no holes—solid telescoping catheter, that is provided over the holes 60 in the hypotube 40 that would block off flow of the treatment solution (through pores 60) to target a specific length of vessel wall 20. Hypotube 40 comprises pores 60.

FIG. 6 shows an exemplary embodiment comprising moving the balloon 10 proximately to prevent blood flow. Vessel walls 20. Additionally, a filter 30 is placed distally to the balloon 10 to prevent emboli, i.e. capture and retain particles so that they do not travel to other places in the body. Hypotube 40 comprises pores.

FIG. 7 shows an exemplary embodiment comprising a proximal balloon 10 to prevent blood flow with a hypotube 40 having perforated sides (pores-holes) to allow drug to be irrigated into the vessels, surrounded by vessel walls 20. Hypotube 40 comprises pores lines 45.

FIG. 8 shows an exemplary embodiment further comprising one or more of a hypotube 40, wire 20 or microcatheter 40 with an occlusive mechanism (or occlusive element) on the distal end, e.g., balloon 10. Further, a method embodiment including a device or system and a treatment solution (including but not limited to a composition comprising DMSO+rapamycin formulation), wherein a radiopacifier 30 is added to the drug formulation as a means of visualizing where the device is located within the body (e.g. vessel wall 50) so that a clinician can identify the area of the vessel that is being treated during the procedure. Additionally included in this embodiment is the feature of a catheter for the evacuation of any remaining thrombi or emboli after the procedure is completed. The catheter could be in purpose as well me too to eliminate the need for a hypotube and be used to deploy the solution and extract or evacuate any remaining blood or remnants of the treatment. The distance of the catheter from the balloon 10 could be adjusted to accommodate for various lesion lengths.

FIG. 9 shows an exemplary embodiment of a device and/or system having a wire 30 inserted distal balloon 20 further comprising a proximal hypotube 40 and/or catheter 50 within an additional balloon 10 for blocking blood from entering the treatment area between the balloons. This second balloon 10 comprising an evacuation tube ensures evacuation of blood within a delivery area so as to provide an active agent (arrows) limited to the target area.

FIG. 10 shows an exemplary embodiment of a device comprising at least 3 balloons (10, 30, 40), located along a wire 50 or hypotube(s) 90, for step 1 (upper diagram): evacuating blood from the target treatment area by inflating the middle balloon 10, and Step 2 blocking of the treatment area by inflating the 2 end balloons 30 and 40 then Step 3 deflating the center balloon 10 then Step 4 backfilling the isolated treatment area 70 with a solution comprising an active agent (dotted lines 80). The idea in this embodiment is to use three balloons in step one of this figure you would inflate the center balloon 10 to evacuate the area 60 between isolated vessel walls 20 which is also between balloons 20 and 30 of any (almost all) blood. The second stage comprises inflating the distal 30 and proximal balloons 40 then deflating the center balloon, while introducing an active agent solution to the center after the blood has been evacuated. In certain embodiments, disruption of the target lesion and retraction of the device, are addressed. In addition, the overall number of lumens that will be used during the procedure will be addressed, based in part upon the type of steps included to deliver a drug for treatment, including parts of the post-treatment procedures, thereby matching the functionality of the device with the contemplated procedure. Thus, in some preferred embodiments, use of the smallest number of lumens per device is desired. However, in other embodiments, depending upon the contemplated steps in combination with a corresponding type of device, additional lumens over the smallest number, may be desired. In addition, steps are taken to limit the length of vessel to be treated, e.g., see FIG. 13. It is not intended that the present invention be limited to the means by which the balloon(s) is/are deflated. In one embodiment, the deflation is done by aspirating the saline solution present in the inflated balloons.

A “lumen” refers to a cavity or channel within a tube or tubular structure, e.g. a blood vessel, a device, etc.

FIG. 11 shows an exemplary embodiment of a device as a schematic showing a system comprising a hollow empty space 40 (hard boundaries) between two balloons 10A (upper left device prior to insertion into vessel), after deployment within a vessel having blood flow 50 surrounded by vessel walls 20 (upper right device within vessel having deflated balloons). The balloons 10A consist of multiple small pores 30, filled with a treatment solution such as Sirolimus solution in either 100% DMSO or in a mixture of DMSO and saline that has little leakage from pores during deployment. The balloons will be simultaneously inflated, just enough that the balloon 10A surface with micropores 30 (depicted as small circles in balloon 10A and shown in an enlarged cone shaped area of device in upper left) tightly sticks to the vessel's wall 20 while a squeezing effect from blood flow volume 50 through the empty space allows the drug solution 60 (as small arrows next to vessel wall 20) to pour out of the inflated balloons onto the surrounding tissue. Absorption of the DMSO in the vessel walls 20 is contemplated to be quick with high delivery efficiency. The space between the two balloons will be enough to allow the blood flow 50 and which will also ensure that the drug solution never (or as little as possible) comes in contact to blood stream and hence no (little substantial) loss due to the flush of blood stream 50 as compared to the conventional DCB. In some embodiments balloons 10A are constructed from materials allowing expansion of the balloon upon inflation. In some embodiments balloons 10A are inflated and deflated with saline through a catheter or hypotube attached to each balloon 10A, not shown.

FIG. 12 shows an exemplary embodiment where instead of using 2 inflatable balloons 10A having pores 30 (micropores) as shown in FIG. 11, boundaries of the hollow middle space are made of very thin and flexible plastic or textile (impermeable to fluids) which responds to the shear pressure generated by the blood flow and its hydrostatic pressure. Once the blood (which was having slow movement due to the restenosis will now enter in with greater force and higher volume similar to how water moves with force when it's released from a dam or restrictions) moves the space between the balloons 10B inflates space 40 and which in turn squeeze the 10B balloons or bags on the ether sides allowing the drug solutions 60 to be delivered to the vessels wall.

In some embodiments balloons 10B are constructed from materials such as flexible plastic. In other words, balloons 10B are squishable balloons, e.g., squishable plastic.

FIG. 13 shows an exemplary embodiment of a method of treatment using a device as described herein for treating thickened vessel wall areas 30, e.g., atherosclerosis, i.e. atherosclerotic plaques, PAD, etc., comprising preparation of the plaque 70 covered vessel wall 30 intended for treatment in the form of restoring blood flow through the plaque either using angioplasty or atherectomy. Thus, in one preferred embodiment, a method of treatment incorporates an angioplasty or atherectomy procedure prior to treatment with a device described herein, for combining within a complete procedure into a method of using the device. In one embodiment, atherectomy procedures include but are not limited to, rotational, transluminal, and directional atherectomy. In one embodiment, atherectomy procedures are used for below the knee applications.

During this type of procedure there are at least two ways to stop the movement of emboli and plaque pieces into the extremities, that arise distal of devices used for treatment. One is filtration of the blood, in other words using a device having an attached filter or using with a device a distal protection filter 90. One is evacuating the disrupted tissue using an evacuation catheter so as not to allow distal movement of the emboli downstream of blood flow. Another is by using a method for treating arthrosclerosis comprising a chemical method of dissolving or removing the atherosclerotic plaque combined with delivering a treatment to the vessel wall in contact with plaque by using a device as described herein. In one embodiment, a device described herein having microholes 80 in the outer surface 20 provides a chemical profusion through the hypotube 20 (infusion through the center of the hydrotube and irrigating into the vessel through the microholes 20 (micropores) depicted here and in FIG. 12. Infusion would happen for a period long enough for the chemical formulation to dissolve the target lesion but short enough so as to not cause clinical issues associated with stopping blood flow. Once sufficient erosion of the target lesion is achieved the fragmented lesion would be evacuated through the evacuation catheter 60, also depicted in FIG. 12. Once the lesion is removed, flow is sufficiently restored, and particulate/emboli are evacuated the drug solution could then be infused into the target area.

Aspects of this embodiment include a hypotube 20 to keep a low profile of the delivery system also enable the crossing through difficult lesions also known as “pushability.” The evacuation catheter 60 with open lumen into the inner section between the two balloons 10 and 40 so that blood and emboli can be removed, and its associated proximal balloon 40 for stopping flow. the restriction catheter 50 to allow for variable length to accommodate different lesion lengths. A restriction catheter may also be used in this system and method in order to allow for variable length of hole 80 coverage 50 in order to accommodate different lesion lengths by covering the holes over areas not intended for treatment. In other words, telescoping a hollow restriction catheter 50 over a hypotube 20 comprising pores 80 allows for variable length expulsion of drug from open holes that are not covered by the surrounding catheter, in order to provide treatment to different lesion lengths using one device/system. Guide wire 100.

FIG. 14 shows an exemplary flowchart in Yang et al. “Endovascular Debulking of Human Carotid Plaques by Using an Excimer Laser Combined With Balloon Angioplasty: An ex vivo Study.” Front Cardiovasc Med., 2021. At least one or more components shown in this chart are contemplated for use herein when evaluating devices, systems, compositions and methods for evaluating treatments of plaque as described herein. In this flowchart, an exemplary carotid plaque at 1 is isolated and placed within a sample holding device at 2. However, it is not intended to limit samples to carotid plaque. Samples may be plaque or arteries containing plaques isolated from other parts of the body including but not limited to lower extremities. In this model, duplicate samples within sample tubes at 3 are treated with an excimer laser at 4, a drug coated balloon at 5 of the present inventions and both at 6, as merely one or more examples.

FIG. 14 Continued shows after treatment, saline from a wash is collected at 1 for staining and observing plaque material in the wash at 2. Treated tissue (plaque) at 3 is evaluated by microscopy, e.g., micro-CT at 4, followed by paraffin embedding at 5 tissue sectioning at 6 (for providing a paraffin section for observation) and staining of section at 7, for additional observation, e.g. micro-computed tomography; and at 8 SEM, scanning electron microscope for observation of tissue at 3, merely for examples of evaluating success of disrupting plaques.

FIG. 15 shows an exemplary SpiderFX™ embolic protection device (Medtronic) comprising a filter 10, wires 20, and catheter 30, that may find use with devices, systems and/or methods as described herein.

FIG. 16 shows an exemplary Cordis/Cardinal Health Angioguard® RX/XP comprising a filter 10 and wires 20, that may find use with devices, systems and/or methods as described herein.

FIG. 17 shows an exemplary Filterwire EZ™ Embolic Protection System of Boston Scientific. Uniform 110-micron-pore filter 10 is designed to permit continuous blood flow while maintaining embolic capture efficiency, that may find use with devices, systems and/or methods as described herein. Inventive device may include components such as radiopaque material in loop 20, suspension arm wire 30, wire 70 inside of catheter 40, catheter stop 50 spinner tube 60, etc. As merely one example, pore size of filter may be 100 microns. Optionally, at the distal end of spinner tube, 80 insertion tip or continuation of wire 70 inside of a catheter 40.

FIG. 18 shows an exemplary schematic of a three-dimensional FiberNet® filter 10 expanded to the vessel wall 20. FiberNet® Embolic Distal filter Protection System of Medtronic may find use with devices, systems and/or methods as described herein. System includes a wire or catheter or hypotube at 40 and a distal filter 10 for filtering out emboli and debris 30, shown in relation to a vessel wall 20. Support structure 50 for holding open vessel, e.g., implantation of stent 50, during a procedure may also find use in the present inventive devices described herein.

FIG. 19 shows an exemplary schematic of a Sentinel® Cerebral Protection System of Boston Scientific Corporation, United States of America, that may find use with devices, systems and/or methods as described herein. System includes a wire 20 and/or catheter 30 or hypotube and a distal filter 10 for filtering out emboli and debris, shown in relation to a vessel wall 20.

FIG. 20 shows an exemplary Emboshield NAV6™ Embolic Protection System of Abbott, United States of America, that may find use with devices, systems and/or methods as described herein. An Emboshield NAV6™ Embolic Protection System includes BareWire™ Filter Delivery Wires, allows the guide wire 20 to rotate and advance freely, independent of the Emboshield NAV6™ filter 10.

FIGS. 21A and 21B shows an exemplary CRE™ RX Balloon Dilatation Catheters Boston Scientific Corporation, that may find use with devices, systems and/or methods as described herein. CRE™ PRO, CRE™ RX, CRE™ Fixed, and CRE™ Wire guided Balloon Dilatation Catheters provide for embodiments of balloon endoscopy for optimal control, efficiency, and performance. Both CRE™ RX Biliary and CRE™ PRO Wire guided Catheters are indicated for use in the removal of difficult biliary stones (Dilatation Assisted Stone Extraction, DASE).

Exemplary devices comprise balloon 10, wire 20 (with or without a curved distal tip), catheter tip 30, injection port 40, indication of overall adjustable catheter/wire length 50 but not a feature at this location, balloon catheter 60, radiopaque material 70, inflation-deflation port 80.

In one embodiment, balloon 10 may be different sizes, e.g., where in one embodiment balloon 10 may be longer than in other embodiments where balloon 10 is shorter.

FIG. 21A shows an exemplary embodiment where wire 20 is encased entirely within catheter 60. FIG. 21B shows an exemplary embodiment where wire 20 is partially encased entirely within catheter 60.

FIG. 22 shows an exemplary Catheter Unibal Balloon—3 Way Silicone Foley Catheter of Haiyan Kangyuan Medical Instrument Co., Ltd., that may find use with devices, systems and/or methods as described herein. The balloon 10 may be inflated with sterile distilled water or 5%, or 10% glycerin sterile aqueous solution. For deflation, cut off the inflation funnel above the valve, or using a syringe without needle push into valve to facilitate drainage.

Exemplary devices comprise balloon 10, wire 20 (with or without a curved distal tip), catheter tip 30, injection port 40, port plug 80, port cap 90 comprising an attachment to port 100, adjustable catheter/wire length indicated at 50 but relates to the entire chosen catheter-wire length, hollow catheter 60, radiopaque material 70.

FIG. 23 shows an exemplary schematic of a Cook Medical's Hercules® 3 Stage Wire Guided Esophageal Balloon, that may find use with devices, systems and/or methods as described herein. Hercules® 3 stage balloons are a strong because they are made of PET plastic—a durable material that has the strength to stand up to a GI (gastrointestinal) stricture with PET. Hercules® balloons not only exert radial force on the stricture but also maintain their position during the dilation procedure. Hercules® 3 stage balloon shows exemplary components which may find use in present inventions. Exemplary devices comprise a prelubricated balloon 10 which facilitates removal from the accessory channel (e.g., catheter or hypotube); balloon 10 allows ability to visualize through the balloon which permits a view of the dilation in progress, example radiopaque material 40; catheter tip 20 is a flexible atraumatic tip that provides access through tight strictures; (guide) wire 30. Rapid balloon deflation is achieved with the express-evacuation catheter 60 and rapid deflation sleeve 70. The kink resistant nitinol catheter 60 provides maneuverability.

FIG. 24 shows an exemplary Medtronic Chameleon™ PTA balloon catheter, that may find use with devices, systems and/or methods as described herein. Exemplary devices comprise components such as a balloon 10, catheter 20, wire 30, injection port 40 for therapeutic fluids, diagnostic imaging and angioplasty, port 50 for balloon inflation and deflation, port for guide wire 60, targeted exit port 70 for releasing fluids into vessel, 80 indicates adjustable catheter/wire length but not an actual component at this location. In some embodiments, guide wire 30 remains in place during procedures.

FIG. 25 shows an exemplary balloon 10 inflation for complete occlusion. In one embodiment, inflate balloon 10 to 1.1 or 1.2 times bigger size to the vessel diameter. In one embodiment, vessel size ranges from 1 mm, 2 mm, 3 mm, up to 4 mm. In one embodiment, an injection volume ranges from 0.01 ml, 0.02 ml, 0.04 mL, 0.06 mL, up to 0.1 mL or more.

FIG. 26 shows the exemplary structures of treatment drugs Rapamycin and Ascomysin.

FIG. 27A shows an exemplary calibration curve preparation of rapamycin. LC spectra of IS.

FIG. 27B shows an exemplary calibration curve preparation of rapamycin. LC spectra of rapamycin.

FIG. 27C shows an exemplary calibration curve preparation of rapamycin. Calibration curve.

FIG. 28 shows an exemplary diagram of popliteal (tibial) arteries through knees into lower extremities, in addition to other exemplary vessels that may be treated as described herein.

FIG. 29 shows an exemplary schematic diagram of one embodiment comprising a full delivery system including a delivery device 101, port 102 for supplying saline through a connected first hypotube 108, comprising a channel, through opening 122 for inflating and deflating angioplasty balloon 116 (which has closed ends); port 104 for supplying saline through a connected second hypotube 110, comprising a channel, for supplying saline through pores and/or micropores 120 into distal balloon 114, where saline passes through pores and/or micropores 120 located at ablated ends 118 for inflating distal balloon; port 106 through a third connected hypotube 112, comprising a channel, for supplying a drug into a vessel, such as an artery, and heat shrink 130. Heat shrink tubing is a thermoplastic tube that shrinks when exposed to heat. When placed around wire arrays and electrical components, heat shrink tubing collapses radially to fit the equipment's contours, creating a protective layer. It is not intended that the present invention be limited to the means by which the balloon is deflated. In one embodiment, the deflation is done by aspirating the saline solution present in the inflated balloons. FIG. 29 shows three different hypotubes (108, 110 and 112) that create three channels.

FIGS. 30A-30E show exemplary methods of using a device 101 embodiment as show in FIG. 29. Port 102 for supplying saline through a connected hypotube 108 through opening 122 for inflating and deflating angioplasty balloon 116 (which has closed ends); port 104 for supplying saline through a connected hypotube 110 for supplying saline through pores and/or micropores 120 into distal balloon 114, where saline passes through pores and/or micropores 120 located at ablated ends 118 for inflating distal balloon; port 106 through a connected hypotube 112 for supplying drug into a vessel, such as an artery. It is not intended that the present invention be limited to the means by which the balloon is deflated. In one embodiment, the deflation is done by aspirating the saline solution present in the inflated balloons.

FIG. 30A shows an exemplary delivery device 101 inserted into an affected vessel, example artery, whose walls in part are represented by 2 parallel black lines surrounding device 101. A deflated angioplasty balloon 116 is located adjacent to target tissue, example where a plaque is present on the blood vessel artery wall.

FIG. 30B shows an exemplary distal balloon 114 inflated for occluding a vessel, such as an artery. Distal balloon 114 is inflating as saline is introduced through port 104 and connected hypotube 110 expelling saline through opening 122 as shown by saline represented by swirl lines inside distal balloon 114.

FIG. 30C shows an exemplary angioplasty balloon 116 is inflated to condense size of target tissue, such as for reducing plaque size. Angioplasty balloon 116 is inflated by saline introduced through port 102 and connected hypotube 108 exiting opening 122.

FIG. 30D shows an exemplary deflated angioplasty balloon 116 inside of a vessel.

FIG. 30E shows an exemplary drug disbursement adjacent to target tissue, such as a plaque. Drug is administered within the through drug port 106 connected to hypotube 112 and pores 120. Administered drug is represented by wavy lines in front of distal balloon 114 and in target tissue areas, such as plaque. Device 101 is removed after drug delivery by deflating distal balloon 114 then removing device 101 from vessel.

FIG. 31 shows exemplary results from the sirolimus calibration curve and encapsulation analysis. The calibration curve ranged from 50 ng/mL-2 g/mL. The absorbance peak at 288 nm corresponds to Sirolimus. A linear curve was obtained upon plotting concentration against the respective absorbance that generated R² value of 0.9999. The calibration curve was utilized to calculate the unknown amounts of Sirolimus.

FIG. 32 shows exemplary scanning electron microscopy (SEM) photographs of Cationic Gelatin Nanoparticles (GNP). Lower magnification upper photograph; higher magnification lower photograph.

FIG. 33 shows an exemplary Sirolimus calibration curve and encapsulation analysis.

FIG. 34 shows exemplary release kinetics of sirolimus from cationic Gelatin Nanoparticles (GNP) under sink conditions.

FIG. 35 shows exemplary cell viability test results: THP-1 cell line results shown in the upper chart and RAW 264.7 cell line results shown in the lower chart. Results show over time and over increasing dosages of empty-cationic Cationic Gelatin Nanoparticles (GNP).

### Definitions

As used herein, “patency” refers to a condition of being open, expanded, or unobstructed.

As used herein, “hypotube” refers to small diameter tubes used in medical applications which typically have one or more hollow channels.

As used herein, “catheter” refers to a tube used in medical applications, which may be solid or have a central open channel, said open channel comprising one or more openings (e.g. holes, pores, etc.)

As used herein, “microcatheter” refers to catheter tubes having 0.70-1.30 mm diameters.

As used herein, “intima” refers to a layer of a blood vessel wall including arteries and veins.

As used herein, “neointima” or neointimal” refers to new or a thickened layer of intima. Neointimal hyperplasia or growth refers to post-intervention, pathological, vascular remodeling due to the proliferation and migration of vascular smooth muscle cells into the tunica intima layer, resulting in vascular wall thickening and the gradual loss of luminal patency which may lead to the return of vascular insufficiency.

As used herein, “growth inhibitors” include any compound that inhibits or reduces growth or hyperplasia. Pharmaceutical composition which have been shown to prevent revascularization (inhibit neointimal growth) include such drugs as Sirolimus, temsirolimus, everolimus, Dexamethasone, Alteplase (TPA). prostacyclin, and paclitaxel.

As used herein, “radiopaque” refers to a substance that is opaque to radiation, e.g., visible in x-ray photographs and under fluoroscopy (opposed to radiotransparent).

As used herein, “radiopacifier” refs to a quality or a property of a material, such as typically dense metal powders, being radiopaque to radiation, such as X-rays.

As used herein, “atherectomy refers to a procedure that utilizes a catheter with a sharp blade on the end to remove plaque from a blood vessel.

As used herein, “endarterectomy” refers to surgical removal of part of the inner lining of an artery, together with any obstructive deposits, carried out on arteries and on vessels supplying the legs.

As used herein, “restenosis” in general refers to narrowing of a blood vessel diameter resulting in restricted blood flow.

As used herein, “Target lesion revascularization” refers to any procedure performed to restore luminal patency after there has been late luminal loss, as one example, loss attributable to in-stent restenosis (ISR).

### DESCRIPTION OF THE INVENTION

The present invention relates generally to medical compositions, methods and devices/systems for treating vascular diseases. More particularly, the invention relates to medical methods, devices and kits for distributing drug to surrounding vascular tissue to treat neointimal growth. More specifically, the described invention is intended to overcome shortcomings of existing treatments for peripheral artery disease (PAD). Devices and methods of the present invention are specifically intended to treat patients with PAD involving the infrapopliteal (tibial) arteries, e.g., patients having a clinical indication for treatment below the knees (e.g., claudication and/or critical limb ischemia (CLI), and patients with complex disease states, for reducing one or more of morbidity; treatment complications in treated patient populations, a reduction in target lesion revascularization (TLR) rates; and a reduction in patients populations requiring any type of leg amputation.

In one embodiment, the present invention contemplates using a solution-based technology wherein a growth inhibiting drug, e.g., Rapamycin®, also known as Sirolimus and Rapamune®, is dissolved in a solvent used in other aspects of medicine as a tissue penetration enhancer, e.g., dimethyl sulfoxide (DMSO). By using DMSO, in some embodiments, drug coating of a balloon with solid (amorphous or crystalline) Rapamycin® (Sirolimus) with its unwanted side effects, described herein, is eliminated. Further, Rapamycin® may be encapsulated into polymeric or lipid nano carriers for better its better solubility into the tissue surface for deep penetration making a vessel wall treatment process more easy, effective (fewer unwanted side effects), realistic, less laborious, and more cost effective than current treatments. In one embodiment, rapamycin is introduced in or on a nanoparticle. In one embodiment, rapamycin is incorporated during nanoparticle production. In one embodiment, rapamycin is encapsulated in a plurality of nanoparticles. In one embodiment, rapamycin is encapsulated in gelatin nanoparticles (GNPs).

DMSO itself has anti-inflammatory properties and is currently used as a solvent for chemotherapeutic drugs. It is successfully used in humans for treating rheumatic, pulmonary, gastrointestinal, neurological, urinary, and dermatological disorders. DMSO further exhibits protective effects in animal models of artery disease, such as middle cerebral artery occlusion, cerebral hypoperfusion-related neuronal death, mercuric chloride-induced kidney injury, and chemical liver injury. A recent report proposed that DMSO reduces ischemic brain damage through both anti-inflammatory and free radical scavenging properties. When applied 70%-90% DMSO will readily pass through the skin. DMSO is known as a tissue penetration enhancer for diffusing many drugs across membranes into tissues and cells, drugs such as morphine sulfate, penicillin, steroids, and insulin. Relief from drugs delivered by DMSO is reported almost immediately and lasts up to 6 hours.

Another fundamental issue with current commercially available mechanisms for delivering an active agent, e.g., drug, to a vascular target tissue is that active agents are typically not water-soluble thus remains in a solid phase when deployed for treatment. Therefore, the kinetics of transferring the drug into the target tissue are not very effective. The inventive composition of an active agent with DMSO, as described herein, is intended to address the fundamental flaws of existing drug coated balloons and stents by putting the drug in the solution phase with DMSO allowing for effective penetration into the target tissue.

Additionally, the invention describes a method of treating variable length lesions. Embodiments of the invention are intended to provide an effective amount of drug needed to treat the target tissue with little drug lost to circulation while penetrating into the vascular tissue to the depth needed for treatment. Embodiments of the invention are also intended to provide consistency of drug penetration into target tissue throughout the length of the lesion.

In order to improve blood flow to downstream tissues from blocked blood vessels, various revascularization methods may be used to bypass blocked areas or to reopen blocked arteries. Artery bypass surgery can be an effective treatment for stenosed or narrowed arteries resulting from atherosclerosis other causes. However, this is a highly invasive procedure which is also expensive and requires substantial hospitalization and recovery time. Mechanical revascularization methods using balloon angioplasty and/or atherectomy, stenting or surgical endarterectomy may be used to open or dilate arteries. As one example, percutaneous translumenal angioplasty (PTA) commonly referred to as balloon angioplasty, is less expensive, less dramatic than bypass surgery.

In addition, effectiveness of balloon angioplasty has improved with the introduction of the stents, which involves the placement of a scaffolding structure within the arteries after treatment by balloon angioplasty. The stent inhibits abrupt reclosure of the arteries and has some benefit in reducing substantial restenosis, resulting in hyperplasia of cells within the vessel wall.

The current standard of care, and most researched treatment for cardiovascular disease and more specifically peripheral artery disease, typically incorporates some type of active agent to inhibit neointimal hyperplasia (PAD). The method for incorporating an active agent, e.g., drug, onto these devices is commonly by coating the outside of a balloon used for treating PAD. These coatings typically incorporate a solid phase of a drug and/or active agent onto the surface of plastic and/or metal devices including outer balloon surfaces, and require other components designed to provide a medical device and drug during the manufacturing process, including modifying surfaces for drug attachment and enable release in the vessel for treatment. A common component for preparing such a medical device is using an excipient for attaching a drug in its solid phase. However, there are many problematic issues associated with incorporating an active agent in this way. Starting with the manufacturing process it's exceedingly difficult to get a consistent and uniform coding and therefore difficult to provide an effective dose on the surface of the device.

During clinical use, the device may have a brittle or fragile coating which leads to additional issues during use. As the device is removed from the package it gets manipulated before and during insertion into a patient which can cause cracks or disruption in the active agent coatings of these devices. As the device is being inserted into the patient and then deployed there are many actions which can also disrupt the coating. As the devices are being deployed, e.g., both stents and balloons, are mechanically manipulated again disrupting the coating causing flaking. Balloons are inflated or otherwise mechanically manipulated again disrupting the coating causing flaking instead of providing a vessel wall targeted treatment comprising a certain effective dose that does not enter the bloodstream for circulation beyond the treatment area.

As mentioned herein, because of the fragility of the coating and the solid phase nature of the drug that is embedded in the coating, a small percentage of the embedded drug ends up on the inside diameter of the target tissue. The remainder of the drug, excipient and other components of the coating that flaked off ends up distal of the target tissue somewhere in the vasculature beyond the treated area. Additionally, mechanical disruption caused by insertion of one or more devices into then through blood vessels until they reach the treatment area, inflation of the balloon, deployment of stents, inure the vessel. Thus, stretch of blood vessels, tissue fracture can create microthrombi and disrupts the target tissue causing particles from the tissue and the target lesion to flow distal into the vasculature where they may block small vessels. In part because of active agents flacking off devices before and during treatment, tissue injury, and loss of active agents to the blood stream, an active agent is applied to a device using higher amounts than necessary, and wastes expensive active agents.

Therefore, one of several goals, as described herein, for effective treatment of vessel walls is to provide a device having an effective amount of active agent as a coating, or a device delivering an effective amount of active agent to the target vessel wall. In a preferred embodiment, the active agent does not enter the bloodstream for circulation beyond the treatment area.

The described invention is intended to address the aforementioned shortcomings of existing treatments. Embodiments include devices specifically intended to treat patients with PAD involving the infrapopliteal (tibial) arteries including patients having a clinical indication for treatment, e.g., Claudication, which literally means “to limp,” is one of the symptoms of lower extremity peripheral artery disease (PAD), critical limb ischemia (CLI) referring to a severe blockage in the arteries of the lower extremities which markedly reduces blood-flow, and patients with complex disease states, e.g., PAD with coronary artery disease (CAD), PAD with Diabetes mellitus. Benefits of using devices, systems and methods described herein are contemplated to include but not limited to, a reduced morbidity and complications in treated patient populations, e.g., showing superior patency at 6 months with a reduction in target lesion revascularization (TLR) rates; a reduction in patients populations requiring any type of leg amputation, etc.

### Exemplary Methods of Using a Device of the Present Inventions.

In one embodiment, a device is provided as shown in FIG. 29.

FIG. 29 shows an exemplary schematic diagram of one embodiment comprising a full delivery system including a delivery device 101, port 102 for supplying saline through a connected hypotube 108, comprising a channel, through opening 122 for inflating and deflating angioplasty balloon 116; port 104 for supplying saline through a connected hypotube 110, comprising a channel, for supplying saline through pores and/or micropores 120 into distal balloon 114, where saline passes through pores and/or micropores 120 located at ablated ends 118 for inflating distal balloon; port 106 through a connected hypotube 112, comprising a channel, for supplying a drug into a vessel, such as an artery, and heat shrink 130.

FIGS. 30A-30E show exemplary methods of using a device 101 embodiment as show in FIG. 29. Port 102 for supplying saline through a connected hypotube 108 through opening 122 for inflating and deflating angioplasty balloon 116; port 104 for supplying saline through a connected hypotube 110 for supplying saline through pores and/or micropores 120 into distal balloon 114, where saline passes through pores and/or micropores 120 located at ablated ends 118 for inflating distal balloon; port 106 through a connected hypotube 112 for supplying drug into a vessel, such as an artery.

FIG. 30A shows an exemplary delivery device 101 inserted into an affected vessel, example artery, whose walls in part are represented by 2 parallel black lines surrounding device 101. A deflated angioplasty balloon 116 is located adjacent to target tissue, example where a plaque is present on the blood vessel artery wall.

FIG. 30B shows an exemplary distal balloon 114 inflated for occluding a vessel, such as an artery. Distal balloon 114 is inflating as saline is introduced through port 104 and connected hypotube 110 expelling saline through opening 122 as shown by saline represented by swirl lines inside distal balloon 114.

FIG. 30C shows an exemplary angioplasty balloon 116 is inflated to condense size of target tissue, such as for reducing plaque size. Angioplasty balloon 116 is inflated by saline introduced through port 102 and connected hypotube 108 exiting opening 122.

FIG. 30D shows an exemplary deflated angioplasty balloon 116 inside of a vessel.

FIG. 30E shows an exemplary drug disbursement adjacent to target tissue, such as a plaque. Drug is administered within the through drug port 106 connected to hypotube 112 and pores 120. Administered drug is represented by wavy lines in front of distal balloon 114 and in target tissue areas, such as plaque. Device 101 is removed after drug delivery by deflating distal balloon 114 then removing device 101 from vessel.

### DESCRIPTION OF PREFERRED EMBODIMENTS

As noted above, treatment of neointimal growth may include a chemical or surgical procedure to first remove a blockage (prior to treatment of the target tissue with the treatment solution). Such surgical procedures include angioplasty.

While embodiments of device, systems, compositions and methods of treatment are not limited to coronary arteries, the following example uses coronary arteries as a model for angioplasty of blood vessels. In fact, these examples of device, systems, compositions and methods of treatment are also intended and preferred for treating PAD.

Angioplasty or percutaneous coronary intervention (PCI) is a procedure used to open blocked coronary arteries caused by coronary artery disease such as atherosclerosis. It restores blood flow to the heart muscle without open-heart surgery. Angioplasty can be done in an emergency setting such as while a patient is having a heart attack. For angioplasty, a long, thin tube (catheter) is put into a blood vessel and guided to the blocked coronary artery. The catheter has a tiny balloon at its tip. Once the catheter is in place, the balloon is inflated at the narrowed area of the heart artery. This presses the plaque or blood clot against the sides of the artery, making more room for blood flow. When blood pressure decreases, intracellular Ca²⁺ concentration decreases causing smooth muscle cells relaxation and arteriolar vasodilation. Ultimately, this endothelium-independent form of myogenic control keeps arteriolar radial wall stress at a stable level and constitutes one of the pathways to control vascular tone.

Therefore, because of disease variation in from patient to patient, in some cases angioplasty (widening arteries) is performed prior to atherectomy (a procedure in which plaque is removed from the inside of an artery) before therapy, including but not limited to Drug Coated Balloon (DCB) therapy. However, because of the potential adverse side effects of angioplasty, some embodiments of the present invention aim to minimize or reduce the need of performing angioplasty to clear plaques. Moreover, because disease variation also results in differing lengths of vessel walls in need of treatment, in one preferred embodiment, a drug delivery device of the present invention may be shortened or lengthened in order to one device to treat multiple patients having differing lengths of desired vessel wall areas. Thus, overcoming the need for having multiple devices of different fixed lengths of treatment areas.

As described herein, a) use of commercially available balloons with small vessels are problematic that might be addressed by using inventive devices and systems as described herein; b) emboli formation during small vessel treatment procedure are a concern that might be addressed by a filter at the distal end; c) hypotubes (with narrow hollow channels) are better than thin wires in terms of strength; additionally can help to deploy the filter as well as deliver a drug formulation using devices, systems, compositions and methods as described herein.

In one embodiment, there is a desire for using one or a limited number of devices and systems for treating different lengths of vessels.

### Treating Atherosclerotic Plaque(s) Before Therapy (Treatment).

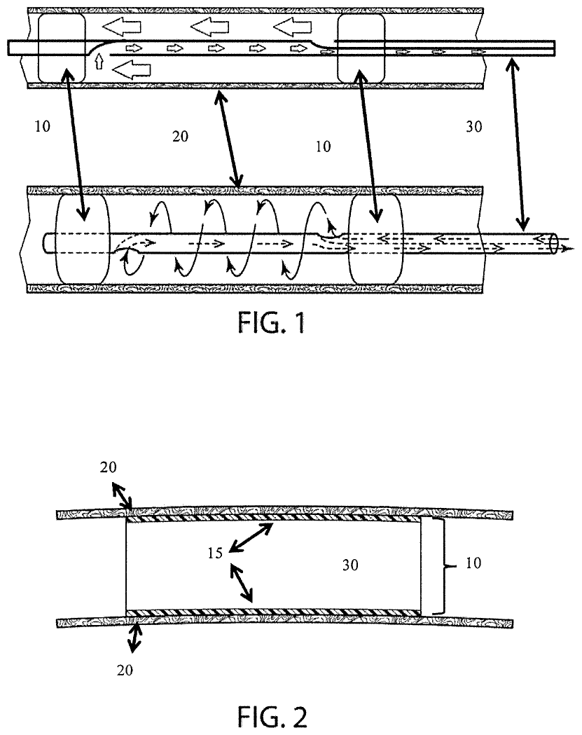

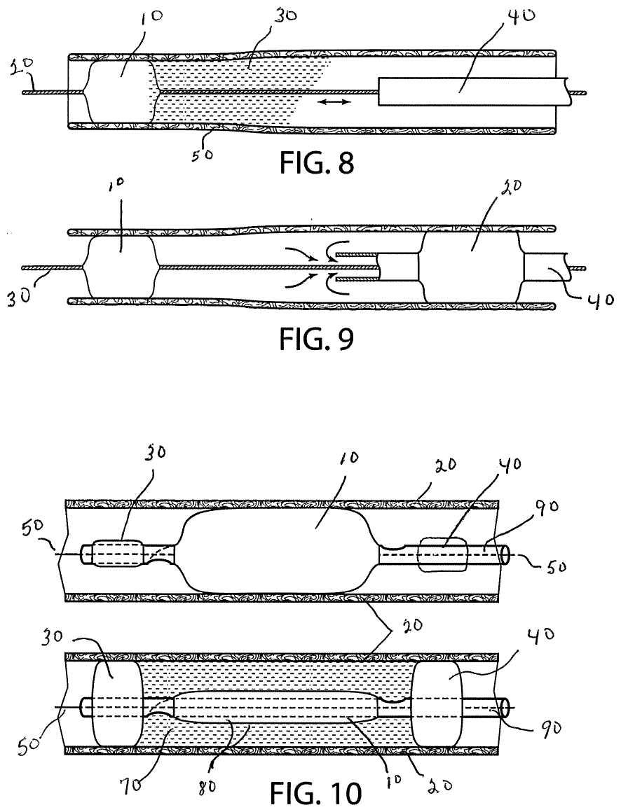

Specific embodiments of a multistep process include (but are not limited to) the following, while inserting and removing medical devices into blood vessels as described below:
1) Occluding blood flow downstream past the target vessel tissue, e.g., stopping blood flow at the distal end. Optionally allowing incoming blood flow at the other, proximal, end. This can be done using a hypotube carrying a filter that is deployed like an umbrella at the distal end (nonlimiting examples of umbrellas shown in FIGS. 15-20);
2) Treating the blockage. This can be done using chemicals, e.g., amine and alcohol-based solvents, chelating agents, Papain enzyme, etc. Alternatively, the blockage can be treated by performing surgical angioplasty or atherectomy, including by devices and methods described herein, including below;
3) Removing particles (potential emboli and cellular debris, etc.) with an evacuation catheter (nonlimiting examples of evacuation catheters shown in FIGS. 8-9);
4) Optionally, performing an additional step of a flush (wash) as described herein (nonlimiting examples of a wash shown in FIG. 1);
5) Introducing a penetration enhancer with a therapeutic agent, e.g., DMSO+rapamycin formulation for delivery to the target tissue (in the presence of blood, since blood flow is not blocked at the other end) for the required period of time (2-30 minutes). This might be done through several nonlimiting means, including introducing fluids through a narrow channel of the hypotube; through balloon assisted drug delivery; or plastic tube drug delivery; etc., as described herein.
6) Removing the entire system from the patient.

The following is one example of a means for step 1 above, occluding blood flow.

### Exemplary Occlusion

### Occlusafe™—Temporary Occlusion Balloon Catheter: Large Lesions Challenges:

Large lesions partially respond to current TACE treatment due to a poor uptake of embolic. Multiple or combined treatments are required to reach a complete response.
Flow Distribution: Through Pressure Gradient Effect, Balloon Occlusion with Occlusafe offers the ability to redistribute blood flow and access the microvascular circulation, leading to an increased accumulation of therapeutics into the target lesion with minimal off-target embolization.
Access to Microvascular Circulation: Balloon Occlusion with Occlusafe™ allows a better visualization for improved diagnosis and higher uptake of embolic into the target lesion.

The following are nonlimiting examples for use in step 2 above, i.e., chemicals for treating blood vessel walls.

### Minimizing or Eliminating the Need of Angioplasty to Clear Plaques Before Therapy (Treatment).

The present invention contemplates, in some embodiments, methods that allow angiopathy to be avoided.

The following sections contain descriptions of nonlimiting options that may be used as alternatives to angioplasty. Such alternatives may be performed as embodiments before or during methods of using Balloon therapy as described herein.

### Calcification Removal Using a Chemical Method

### Example 1. Using Amine and Alcohol Based Solvents

Phospholipid is known to play an important role in bovine pericardium in in vivo calcification. The Ca²⁺ molecules in extracellular fluid are assumed to combine with phosphorus molecules in the phospholipid, which is abundant in dead pericardial cell membranes and forms calcium phosphate crystal. This means that the phospholipid material can be a nidus (a place in which something develops or is fostered) for calcification.

A study was conducted to evaluate the effect of treatment with amino compounds and alcohol-based solvents in vivo on calcifications of glutaraldehyde (GA)-fixed pericardium. Amino compound treatment alone resulted in a dramatic decrease in the Ca²⁺ and inorganic phosphate (IP) concentration. Amine based compounds used were urazole and glutamate and some solvents (ethanol with octanol or octanediol) to reduce the phospholipid content in the bovine pericardial tissue. Anti-calcification treatment with glutamate, urazole, and solvents did not worsen the physical properties of bovine pericardium, and significantly prevented in vivo calcifications. (Interact Cardiovasc Thorac. Surg. 2011 June, 12(6), 903-7; doi: 10.1510/icvts.2010.259747.)

### Example 2. Using Chelating Agents

Chelating agents, such as disodium ethylene diamine tetra acetic acid (EDTA), diethylene triamine penta acetic acid (DTPA) and sodium thiosulfate (STS) can reverse elastin calcification by directly removing calcium (Ca²⁺) from calcified tissues into soluble calcium complexes. The chelating ability of EDTA, DTPA, and STS on removal of calcium from hydroxyapatite (HA) powder, calcified porcine aortic elastin, and calcified human aorta were studied. The tissue architecture was not altered during chelation. In the animal model of aortic elastin-specific calcification, it was further shown that local periadventitial delivery of EDTA loaded into poly (lactic-co-glycolic acid) (PLGA) nanoparticles regressed elastin specific calcification in the aorta. Collectively, the data indicate that elastin-specific medial vascular calcification could be reversed by chelating agents. (Calcif Tissue Int. 2013 November; 93(5); doi: 10.1007/s00223-013-9780-0)

### Example 3. Papain Enzyme

Papain is an enzyme extracted from Carica papaya, such as the product sold under the trade name “Papase' by Warner-Chilcott Laboratories, a division of Warner-Lambert Company, Morris Plains, N.J., 07950. The papain will not affect bone calcium within a living body but does dissolve inert calcium by removing completely, abnormal inert deposits of calcium located be neath the skin of an animal or other living organism, by liquifying or dissolving the calcium and then utilizing the natural circulatory process to remove the irritating calcium deposits from joint or tissue areas without the necessity for surgical treatment or hypodermic injections which might cause further damage. This ingredient also reduces tumescence, aids the flow of blood to affected areas, and assists the natural body circulatory process in bathing those areas. (Acta Orthop. Scand. 1977, 48(2), 143-9; doi: 10.3109/17453677708985125.)

The following are additional means to improve blood flow.

### 1. Peripheral Vasodilators:

Peripheral vasodilators refer to medicines that are used to treat conditions that affect blood vessels in outer (peripheral) parts of the body such as the arms and legs. For example, they are used to treat peripheral arterial disease and Raynaud's phenomenon. They ease the symptoms of these conditions by dilating the blood vessels, preventing them from becoming narrower (constricting). These medicines are usually prescribed after self-help measures have been tried without improving symptoms.

Other means of vasodilation are described in a published study showing treatment options for chronic thromboembolic pulmonary hypertension (CTEPH), not amenable to thromboendarterectomy or recurrent/persistent after thromboendarterectomy (i.e., inoperable CTEPH). Treatment options include pulmonary vasodilators or balloon pulmonary angioplasty (BPA). Kalra et al. 2020. These authors compared efficacy and safety outcomes of BPA with or without pulmonary vasodilators to pulmonary vasodilator therapy alone in patients with inoperable CTEPH. Observational and randomized trial data reporting outcomes for >5 patients with inoperable CTEPH were sought. Single-arm random effects meta-analyses were performed. These authors concluded that BPA and pulmonary vasodilators both improve functional and hemodynamic outcomes in patients with inoperable CTEPH. While BPA may offer greater functional and hemodynamic improvements, this technique carries the accompanying risks of an invasive procedure. More high-quality randomized data with long-term follow-up, and use in more patients, is needed to definitively examine a role of BPA and pulmonary vasodilators for beneficial treatment of patients having inoperable CTEPH.

However, one issue against using vasodilators in general are their transient effects. Further, there is a need for a vessel wall therapy that will disrupt plaques quickly rather than having to open the artery on temporary basis to remove plaques, and to avoid multiple times an artery must be opened to remove plaques from the same areas.

### 2. Chemical Treatment:

In this embodiment, an exemplary vessel wall treated by DMSO is thoracic aorta. Debons et al. investigated the effect of dimethyl sulfoxide (DMSO) on cholesterol-induced atherosclerosis in rabbits. Rabbits on an atherogenic diet which did not receive DMSO had extensive aortic lesions covering 82±5% of the surface area of the thoracic aorta. Aortic lesions were inhibited by about 50% in rabbits on 2% (dose, 1.5 g/kg) DMSO and virtually absent in the majority of rabbits on 4 (dose, 3.5 g/kg), 5 (dose, 5.5 g/kg) and 6% (dose, 9.1 g/kg) DMSO. The food intake of rabbits on the atherogenic diet was not suppressed by DMSO. Debons, et al., J Pharmacol Exp Ther. 1987 November; 243(2):745-57.

### 3. Laser Angioplasty:

Laser Angioplasty is used for treating Critical Ischemia patients that are poor candidates for bypass surgery. One report is a study that concluded laser-assisted angioplasty was highly effective in limb salvage and revascularization of patients who were unfit for bypass surgery. Chen et al. Chapter 11—Laser Atherectomy, Endovascular Surgery (Fourth Edition) 2011, Pages 107-115. Available online 27 Dec. 2010. More specifically, fourteen sites in the United States and Germany enrolled 145 patients with 155 critically ischemic limbs. Treatment included laser atherectomy followed by balloon angioplasty with optional stenting. Stents were implanted in 45% of limbs. At 6-month follow-up, limb salvage was achieved in 110 (92%) of 119 surviving patients or 93% (118/127) of all limbs. Another report is in Yang et al. “Endovascular Debulking of Human Carotid Plaques by Using an Excimer Laser Combined With Balloon Angioplasty: An ex vivo Study.” Front Cardiovasc Med., 2021, which studied safety and effectiveness of applying an excimer laser for debulking human carotid atherosclerotic plaques by investigating the distal debris, plaque luminal gain, and micromorphology of the plaque surface. See, for example an exemplary flow diagram shown in FIG. 14. The authors in Yang, showed that treatment Group 3 (laser+balloon) produced the highest luminal gain (5.40±4.51 mm²), while the other two groups had gains of 4.05±3.20 and 3.77±2.55 mm² respectively. Both devices caused disruptions to the plaque lumen surface. Moreover, laser ablation exposed fibers under the endothelium, i.e. disrupting endothelium, while balloon angioplasty cracked the surface of blood vessels. Mean amounts of damage were 3,611±1,475.4 for group 1 (laser only), 2,828±1,266.7 for group 2 (balloon only), and 4,400±2,567.9 for group 3 (laser+balloon). More than 90% of the distal debris was smaller than 10 m. Group 2 produced the most debris with Feret (maximum caliper diameter) >40 m; group 1 had the least. The author concluded that excimer laser ablation could significantly increase the luminal gain of carotid plaque with high stenosis. Excimer laser combined with balloon angioplasty achieved the highest lumen enlargement. 4. Coronary Artery Bypass Surgery:

Coronary artery bypass surgery, also known as coronary artery bypass graft (CABG) surgery, and colloquially heart bypass or bypass surgery, is a surgical procedure to restore normal blood flow to an obstructed coronary artery. A normal coronary artery transports blood to the heart muscle itself, not through the main circulatory system. CABG is often indicated when coronary arteries have a 50 to 99 percent obstruction. The obstruction being bypassed is typically due to arteriosclerosis, atherosclerosis, or both. Arteriosclerosis is characterized by thickening, loss of elasticity, and calcification of the arterial wall, most often resulting in a generalized narrowing in the affected coronary artery. Atherosclerosis is characterized by yellowish plaques of cholesterol, lipids, and cellular debris deposited into the inner layer of the wall of a large or medium-sized coronary artery, most often resulting in a partial obstruction in the affected artery. Either condition can limit blood flow if it causes a cross-sectional narrowing of at least 50%. Cleveland Clinic: Coronary Artery Bypass Surgery (my.clevelandclinic.org/health/treatments/16897-coronary-artery-bypass-surgery). Downloaded 2-18-2022.

### 5. Reperfusion Therapy:

Reperfusion therapy refers to a medical treatment to restore blood flow, either through or around, blocked arteries, typically after a heart attack (myocardial infarction (MI)). Reperfusion therapy includes drugs and surgery. The drugs are thrombolytics and fibrinolytics used in a process called thrombolysis. Surgeries performed may be minimally invasive endovascular procedures such as a percutaneous coronary intervention (PCI), followed by a coronary angioplasty.

Angioplasty is used as an insertion of a balloon to open up blocked arteries, with the possible additional use of one or more stents. Other surgeries performed are the more invasive bypass surgeries that graft arteries around blockages.

Barron et al., concluded that the reperfusion therapy, with either the administration of a thrombolytic agent or immediate angioplasty, is clearly a beneficial therapy for patients presenting with a myocardial infarction, yet it remains underutilized. Their data suggest that of those patients eligible for reperfusion therapy, 24% do not receive this proven therapy. Specifically, women, the elderly, patients without chest pain, and those patients at highest risk for in-hospital mortality were least likely to receive reperfusion therapy. In order for reperfusion therapy to realize its full potential in reducing cardiovascular mortality, the translation of the findings of randomized controlled trials into clinical practice must occur. Barron et al., Circulation Volume 97, Issue 12, 31 Mar. 1998; Pages 1150-1156.

### 6. Filter at the Distal End of a Medical Device:

Embolic protection devices (EPDs) were developed to help prevent embolization during endovascular procedures. The risk of distal embolization is considered significant in the carotid arteries, saphenous vein grafts and thrombotic lesions affecting patients with acute coronary syndromes. While EPDs have been designed and clinically tested for these procedures, their use during procedures in the other vascular territories has been questioned because of the increased cost, potential risk of complications and perceived lack of significance of distal embolization in these vascular beds. Some of the commercially available EPDs are described herein and shown in FIGS. 15-20.

The following are nonlimiting examples for use in step 5 above, introducing a penetration enhancer, e.g., DMSO+rapamycin formulation, etc.

### Concentration of DMSO for Rapamycin Delivery

Rapamycin is found to be most stable in DMSO for many months at −20° C. The rapamycin solution in DMSO could be further diluted either in saline, PBS, or distilled water. The current data shows that the lowest concentration of DMSO in saline that allows rapamycin to remain soluble (no precipitation or crashing out of solution) is 20% of the total volume (v/v).

It has been found that the mixing of rapamycin solution in DMSO in saline has to be in a particular order in order to not precipitate the rapamycin out of the solution, this applies to the system where the DMSO is either 20% or lower of the final volume after dilution. It is suggested that the saline is added to the rapamycin solution in DMSO and not the otherwise. The best working solution is considered to be 50/50 DMSO/water i.e., 50% of the DMSO in solution. This could be envisioned valid if instead of saline blood is used.

### Other Chemicals as a Penetration Enhancer

### 1. N-Vinyl Pyrrolidone (NMP)

N-methyl-2-pyrrolidone (NMP) is a polar aprotic solvent and miscible with most common solvents including water and alcohols. NMP can partition well into human stratum corneum. Within the tissue, it may act by altering the solvent nature of membrane and has been used to generate “reservoirs” within skin membranes. It was also used as skin penetration enhancer in many topical formulations at the concentration up to 40% without any skin sensitization. (Asian journal of pharmaceutical sciences 8 (2013) 110-117; doi: 10.1016/j.ajps.2013.07.014)

### 2. Ionic Liquids:

Currently, ionic liquids (ILs) are a class of compounds under intensive investigation for biomedical applications—more specifically transdermal drug delivery. Based on the reported use of ILs as chemical permeation enhancers (CPEs), there is continued interest for ILs in transdermal drug delivery. ILs were shown to enhance transdermal transcellular and paracellular transport, bypassing the barrier properties of the stratum corneum (SC), employing mechanisms such as disruption of cellular integrity, fluidization, and creation of diffusional pathways and extraction of lipid components in the SC. (Pharmaceutics 2019, 11, 96; doi:10.3390/pharmaceutics11020096 and International Journal of Pharmaceutics 516 (2017) 45-51; doi: 10.1016/j.ijpharm.2016.11.020)

Professor Samir Mitragotri's lab has extensively shown that the ionic liquid/deep eutectic solvent comprised of choline and geranic acid (CAGE) exhibit characteristics that make it a potential candidate for effective treatment of rosacea. The CAGE has been shown to exhibit deep penetration into the skin (Bioengineering & Translational Medicine, 6(2); doi:10.1002/btm2.10191 and Advanced Materials, 1901103; doi:10.1002/adma.201901103. The same lab also synthesized various other ionic liquid/deep eutectic solvent system for deep tissue penetrations application (PNAS, 2014, 111 (37) 13313-13318; doi: 10.1073/pnas.1403995111).

### 3. Lipids and Liposomes:

Poorly water-soluble drugs are challenging for the formulation scientists with regard to solubility and bioavailability. Lipid and liposome-based drug delivery systems (LBDDS) have shown the effective size dependent properties, so they have attracted a lot of attention. Also, LBBDS have taken the lead because of obvious advantages of higher degree of biocompatibility and versatility. These systems are commercially viable to formulate pharmaceuticals for topical, oral, pulmonary, or parenteral delivery. Lipid formulations can be modified in various ways to meet a wide range of product requirements as per the disease condition, route of administration, and also cost product stability, toxicity, and efficacy. Lipid-based carriers are safe and efficient hence they have been proved to be attractive candidates for the formulation of pharmaceuticals, as well as vaccines, diagnostics, and nutraceuticals. (Journal of Pharmaceutics Vol. 2014; doi: 10.1155/2014/801820, Therapeutic Delivery, 2(11), 1485-1516; doi: 10.4155/tde.11.105, Int. J. Mol. Sci. 2020, 21, 3248; doi: 10.3390/ijms21093248, and Front. Pharmacol.; doi: 10.3389/fphar.2015.00286)

Systems of the present inventions include but are not limited to the use of several medical devices during a method of treatment. Merely as one example, in one embodiment, a system for treatment of PAD comprises a wire deploying a distally located filter in addition to a hypotube surrounded by a balloon for introducing an active agent while using a catheter for stopping proximal blood flow, as described herein.

In one embodiment, a device is provided comprising two balloons, proximal and distal, for delivering a treatment solution (such as a DMSO+a drug formulation, e.g., a DMSO+rapamycin formulation) directly to the wall of the vessel while preventing or reducing mixing formulation with blood. After balloons are deployed, an inflow of fluid through a hollow tube through one of the balloons for introduces fluids such as a drug formulation, washing fluids, etc., through the isolated space between the balloons. Conversely, fluids may also be removed, such as blood, drug formulation after treatment, wash fluids, etc. Exemplary FIG. 1.

In one embodiment, devices provided herein maximize the amount of space the balloon takes up in the inside diameter of the vessel. This would allow the DMSO plus a drug formulation to be present against the wall of the target vessel minimizing the exposure of the formulation to the bloodstream. Alternatively, in one embodiment, devices provided herein minimize void space in a balloon. Exemplary FIG. 2. In certain embodiments, the variation in the length of the vessel could possibly be treated as well as the number of lumens required to inflate the balloon and deliver the drug, are addressed. In addition, steps are taken to deal with any emboli created from the inflation and deployment of the balloon as relates to the target tissue.

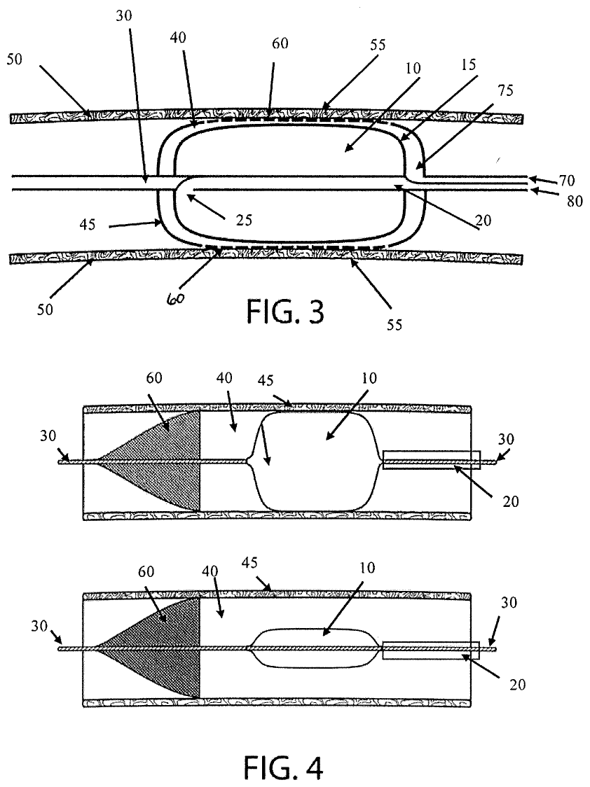

In one embodiment, a method is provided for delivering a DMSO+a drug formulation, e.g., a DMSO+rapamycin formulation directly to the wall of the vessel while preventing or reducing mixing formulation with blood using a balloon within a balloon, where the outer balloon has pores (holes) for introducing DMSO+a drug formulation directly onto vessel walls. Balloons may be inserted with a wire or hollow hypotube. Step 1 is evacuating blood. Exemplary FIG. 3.

In certain embodiments, disruption of the tissue and potential creation of emboli during deployment, are addressed. In addition, steps are taken to limit the length of vessel to be treated. In further embodiments, smaller diameters of vessels less than 4 millimeters would have a delivery system that would sufficiently deliver an active ingredient formulation. Loss of the formulation and generation of emboli when removing the device post treatment, both issues with most balloon-based delivery systems, are addressed.

In one embodiment, a low-profile system comprises a small balloon or other mechanical occlusion feature attached to the distal or proximal end of a guide wire or hypotube to stop or inhibit further blood movement within the vessel. In this embodiment, blood flow is inhibited or stopped until after releasing the treatment solution (including but not limited to a DMSO+rapamycin formulation) into the bloodstream for a specified time period. As the treatment solution mixes with the blood upstream from the occlusion feature, vessels may be treated in the stopped blood, for one example, a time period of between 2 to 30 minutes while the drug formulation is readily absorbed by the vessel target area. This type of embodiment has several distinct advantages including 1) a low-profile system through use of a small hypotube as a wire for delivering the balloon/delivery system to the target site and 2) essentially has the ability to deliver a delivery system to the target vasculature regardless of the size or length of the vessel merely by changing where the balloon is inflated along the hypotube wire. The ability to treat very small vessels with minimal disruption to the vasculature and therefore minimizing any potential emboli created during the procedure itself is one advantage of using this method. Exemplary FIG. 4.

Additionally, in this embodiment, is the presence of a filter on the distal end could be used for multiple beneficial results including preventing any emboli created during deployment of the balloon or created from the DMSO drug combination from entering the blood stream during or after treatment while the device is being removed.

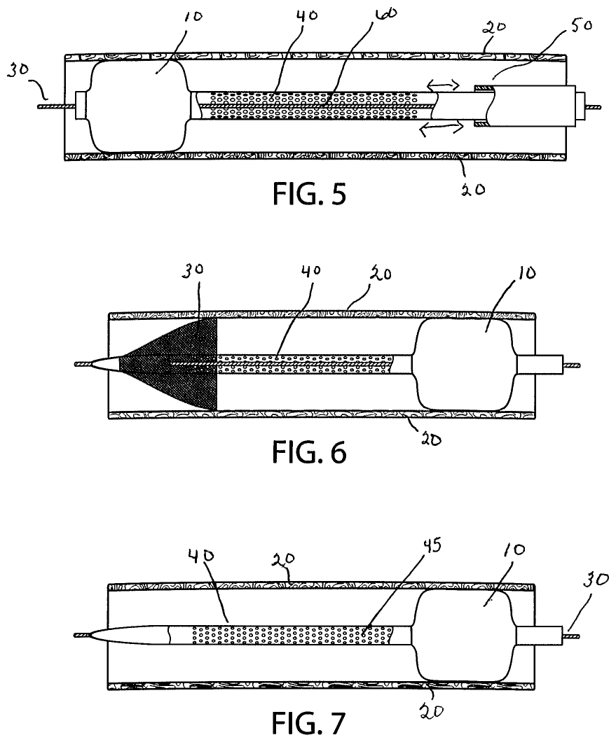

In one embodiment, a device comprises a combination of a wire and a hypotube attached to a balloon or other mechanical occlusion device to the distal end. A small hypotube surrounding a wire would be used to deliver the drug through a plurality of holes (pores), hypotube, i.e. perforated. As the drug is flowed through the hypotube it would elude into the vasculature including the blood. An additional feature with this device is a means to change the length or distance that the drug is being introduced into the vasculature, therefore allowing for treating lesions of different lengths using the same device. In a further embodiment, an additional tube, e.g., catheter, is provided for surrounding a portion of the perforated hypotube for covering over some of the holes in the hypotube that would block off flow of the treatment solution to avoid treating that section of vessel, and in turn target treat a specific length of vessel. Exemplary FIG. 5.

In one embodiment, the balloon is moved proximately to prevent blood flow. In some embodiments, a filter is placed distally from the balloon in order to prevent emboli. Exemplary FIG. 6.

In one embodiment, a balloon is located proximally to prevent blood flow while a distal hypotube with perforated sides allows drug to be irrigated into the vessels. Exemplary FIG. 7. In one embodiment, a device and/or system further includes one or more of a hypotube, wire, balloon, with a microcatheter as an occlusive mechanism on the distal end of a treatment area, with a balloon occluding the proximal end of the treatment area. In a further embodiment, a catheter pushes a treatment formulation into the vessel area targeted for treatment. In some embodiments, a radiopaque, e.g., iodine in fluid may be added to the DMSO+rapamycin formulation, and pieces of radiopaque material attached to wires, catheters, inside balloons, etc., providing a means for a clinician to clearly identify the area of the vessel being treated during the procedure using x-rays or other radiation emitting equipment. Radiopaque materials include but are not limited to small molecular weight salts or compounds or nanoparticles containing iodine, barium, tantalum, bismuth, or gold. Exemplary FIG. 8.

Additionally included in a device embodiment is a catheter for the evacuation of thrombi or emboli after the treatment procedure is completed. The catheter might also be used in place of a hypotube for deploying fluids, e.g., drug solution, and extracting and/or evacuating remaining blood within the treatment area or remnants of the treatment, e.g., emboli, debris, etc. The distance of the catheter from the balloon would be adjusted to accommodate for treating lesions of different lengths as described herein.

In one embodiment, a device comprises embodiments described for FIGS. 3-7, further comprising an additional feature of a balloon surrounding a hypotube or catheter for methods ensuring evacuation of blood, drug delivery to a target vesicular wall area and removal of drug solution after treatment. Exemplary FIG. 9.

In one embodiment, a device comprises 3 balloons along a wire, wherein the proximal balloon surrounds a tube, for use in a treatment method by inflating the center balloon to evacuate blood in an area designated for treatment, followed by inflating the 2 end balloons to block off the vessel treatment area, followed by deflating the center balloon while backfilling the vacant space with a fluid introduced through the tube, e.g., drug solution. In other words, First stage: step 1: inflate the center balloon to evacuate the area of any blood, step 2 inflate the distal and proximal balloons, a second stage would include deflating the center balloon and introducing drug formulation within the center areas (hatched), after the blood has been evacuated. Exemplary FIG. 10.

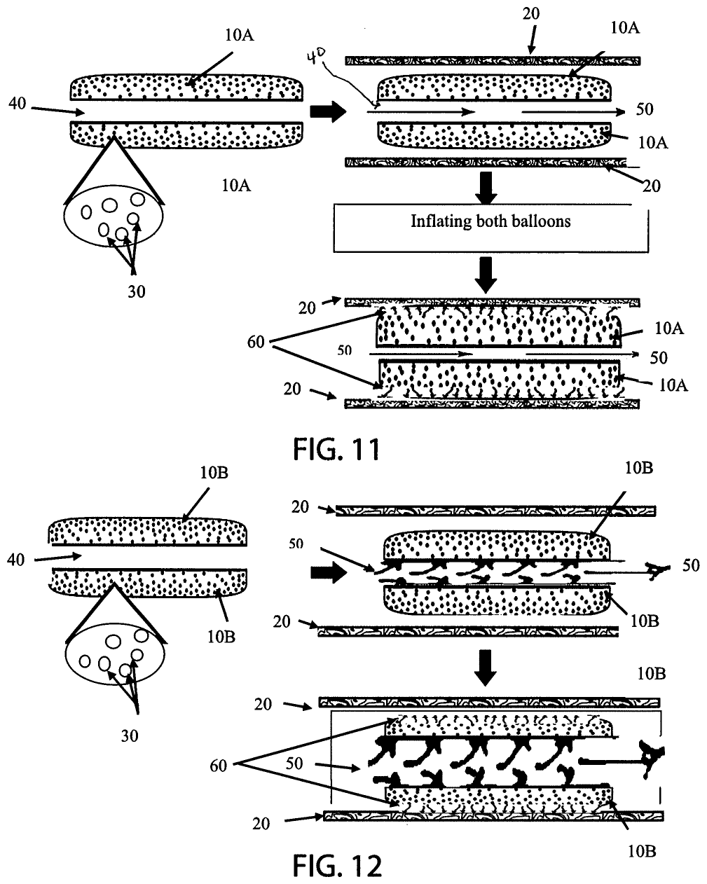

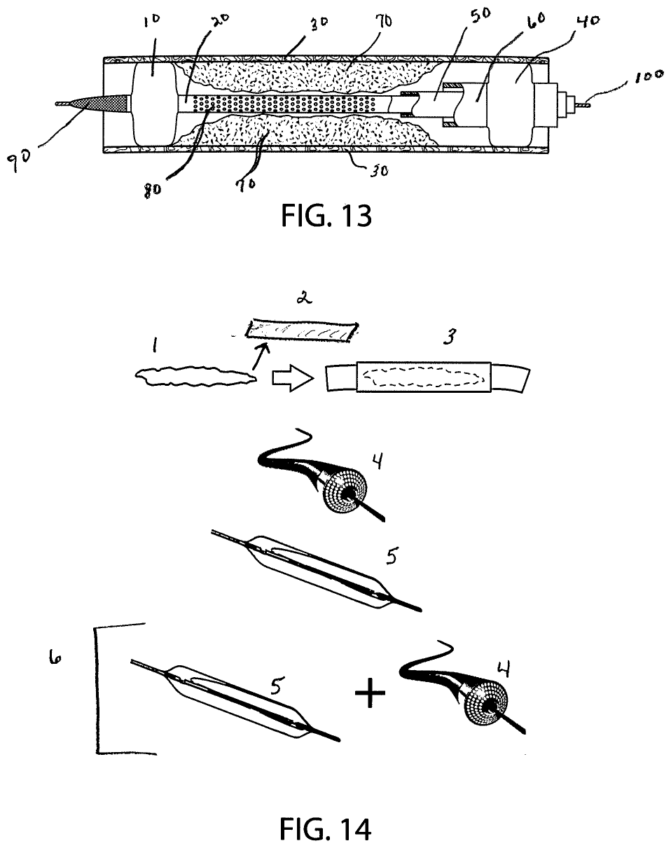

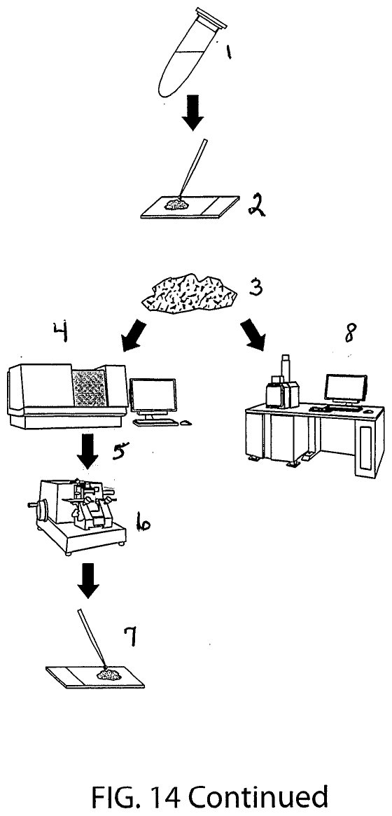

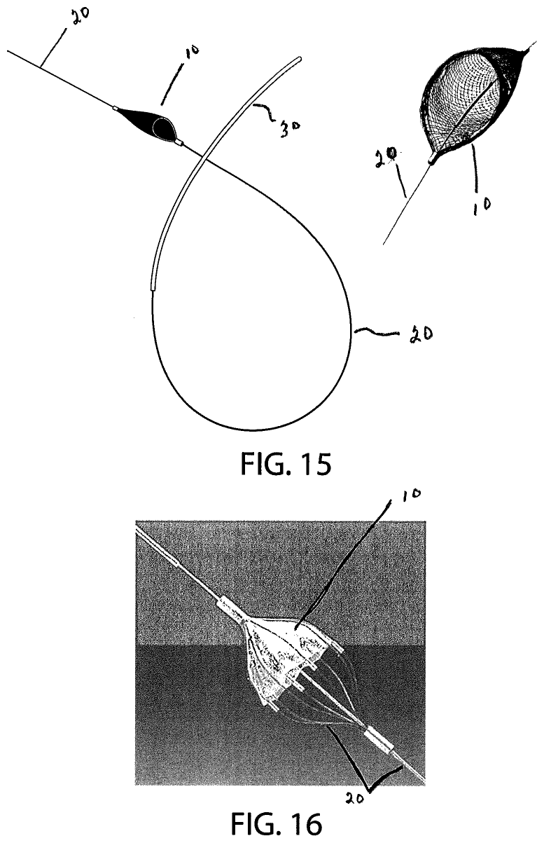

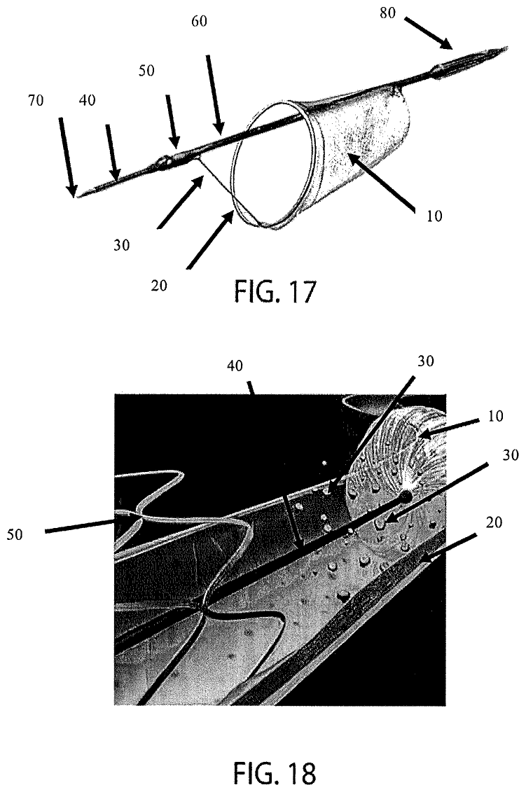

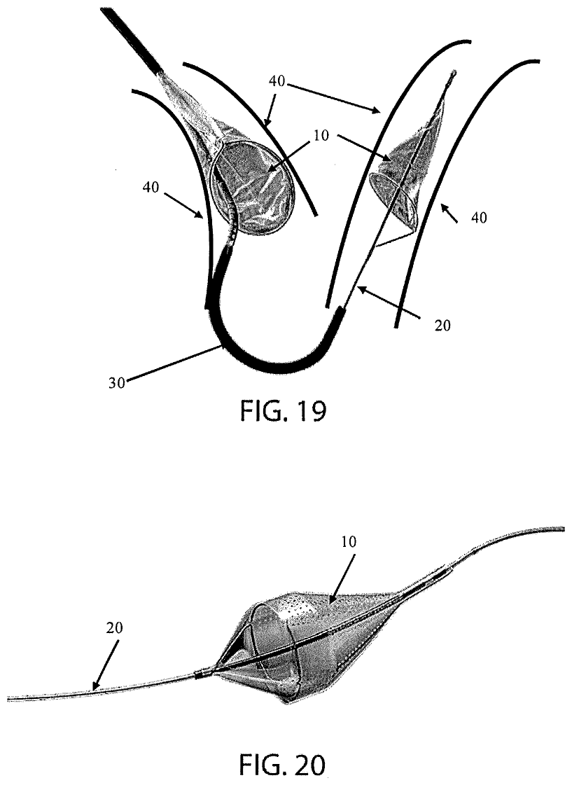

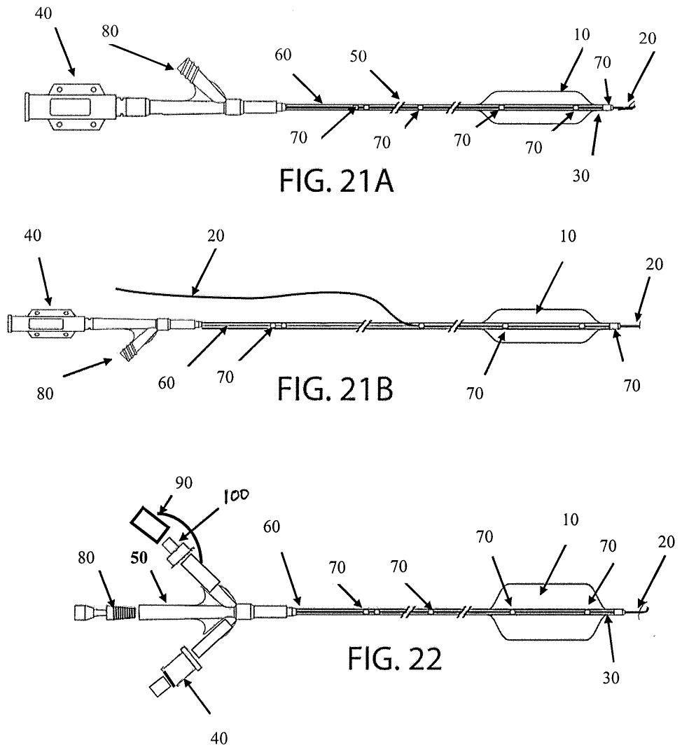

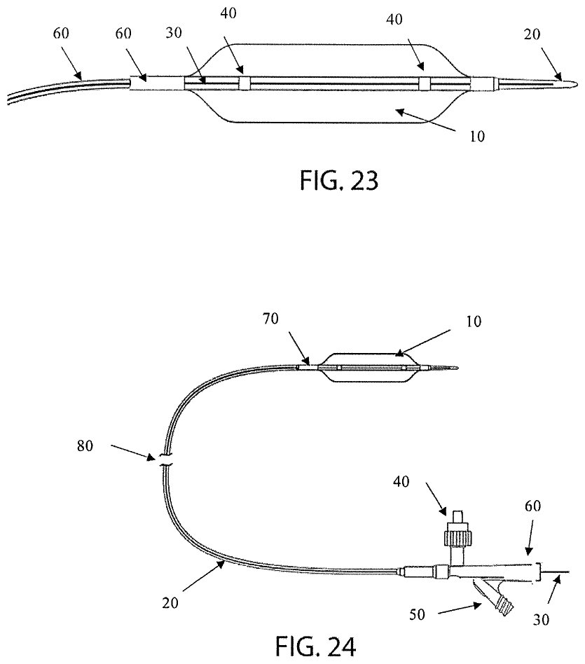

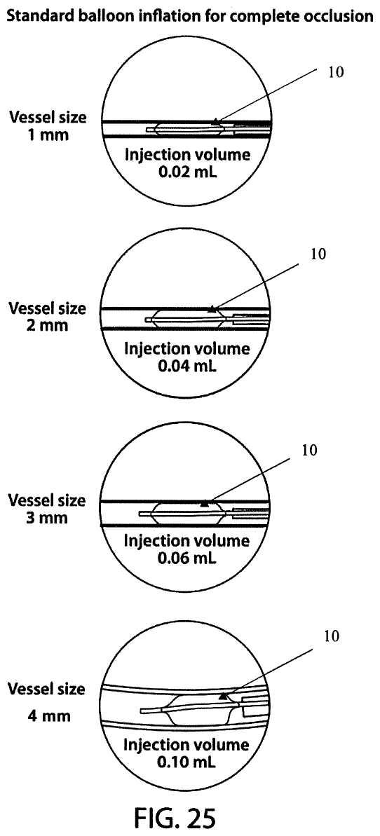

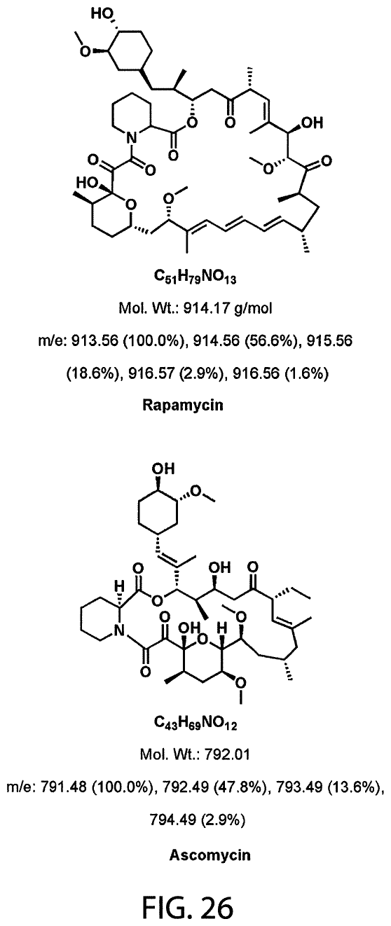

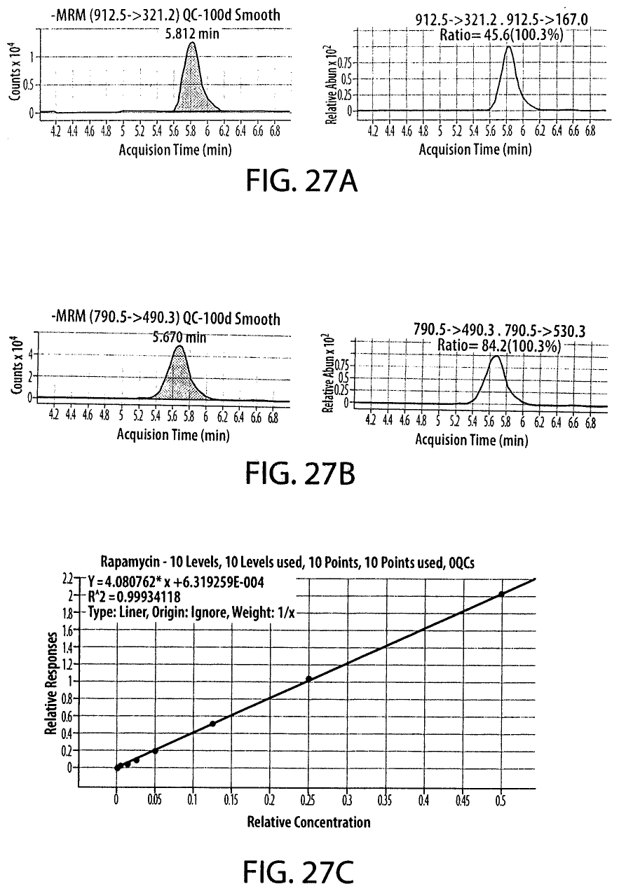

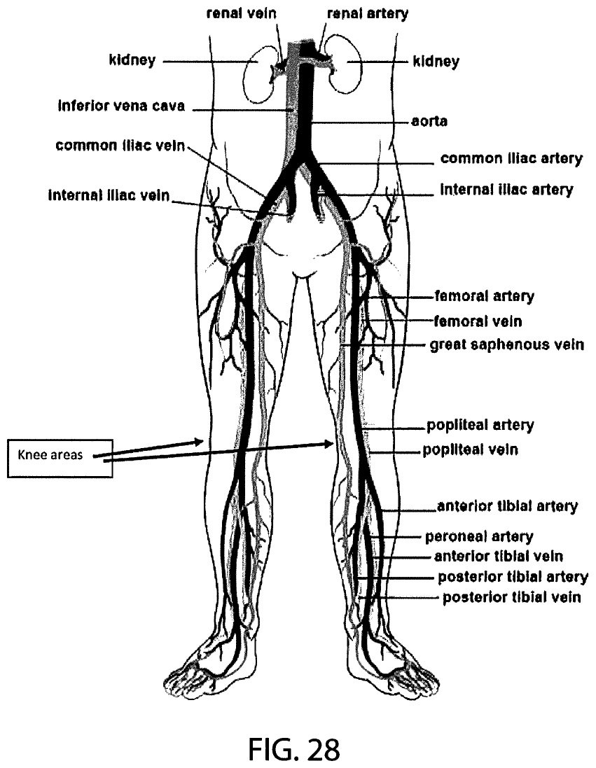

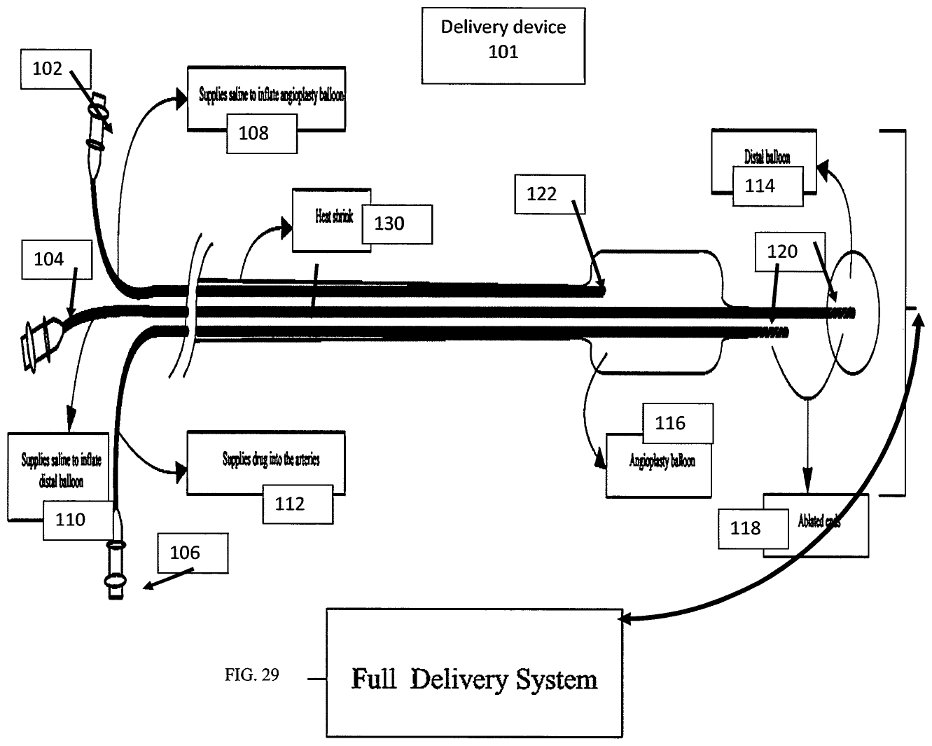

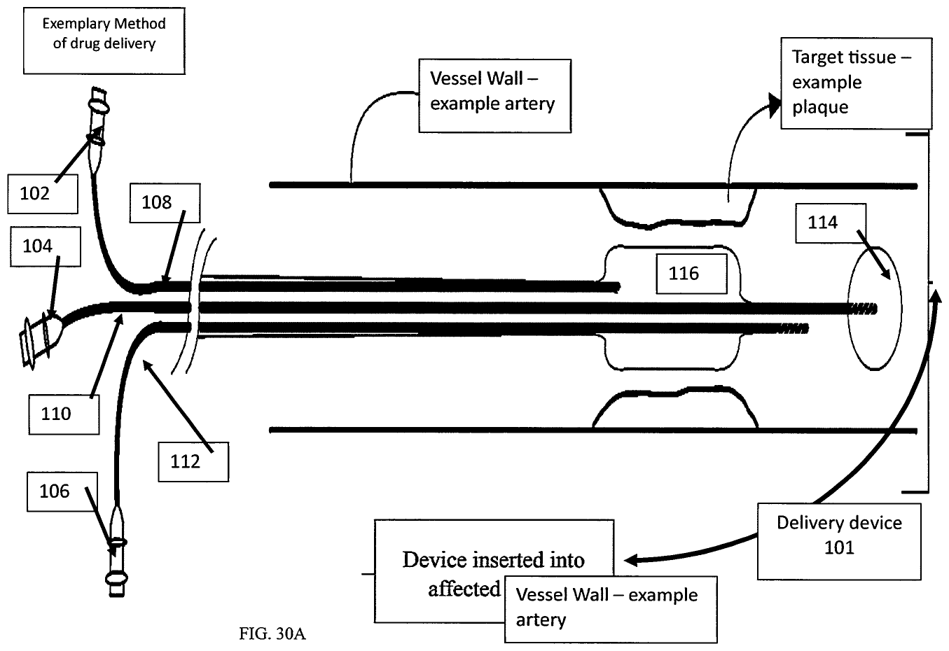

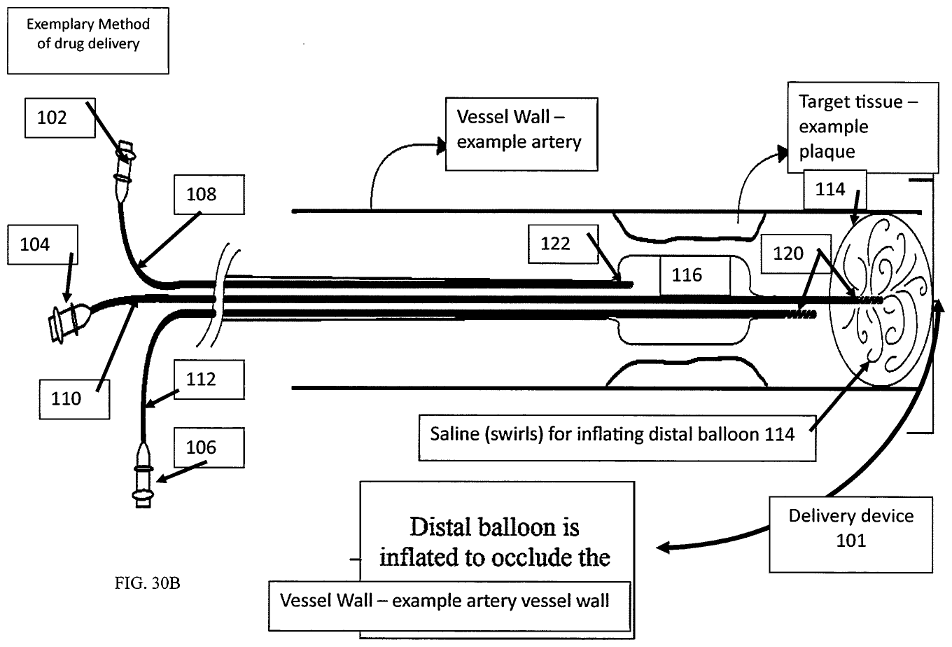

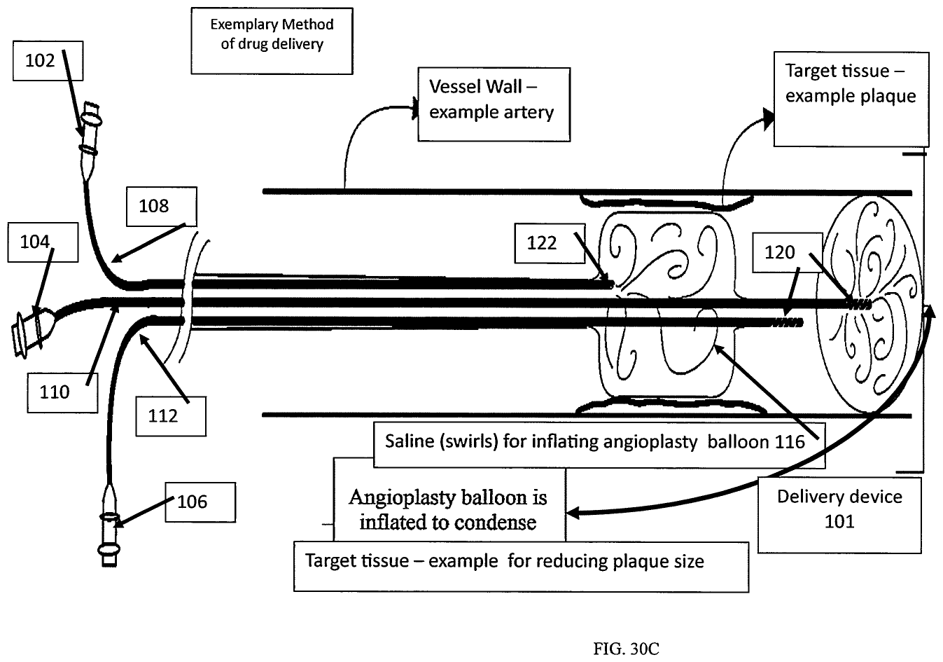

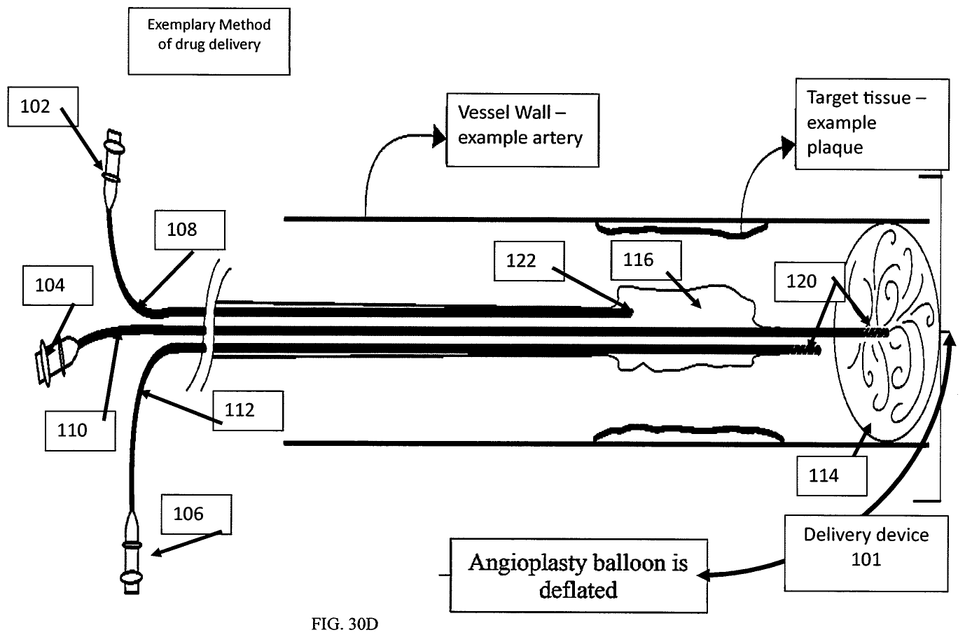

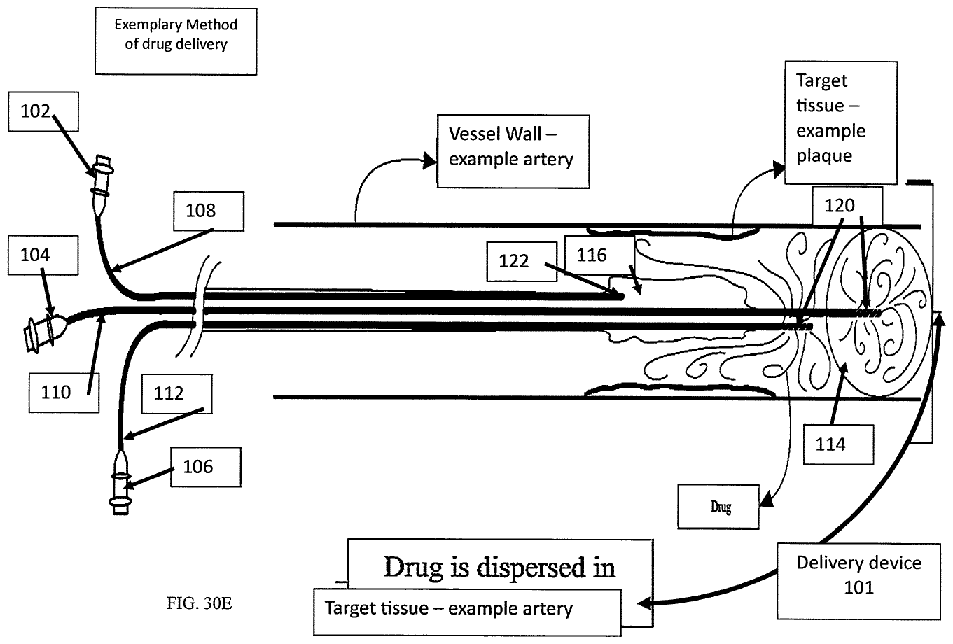

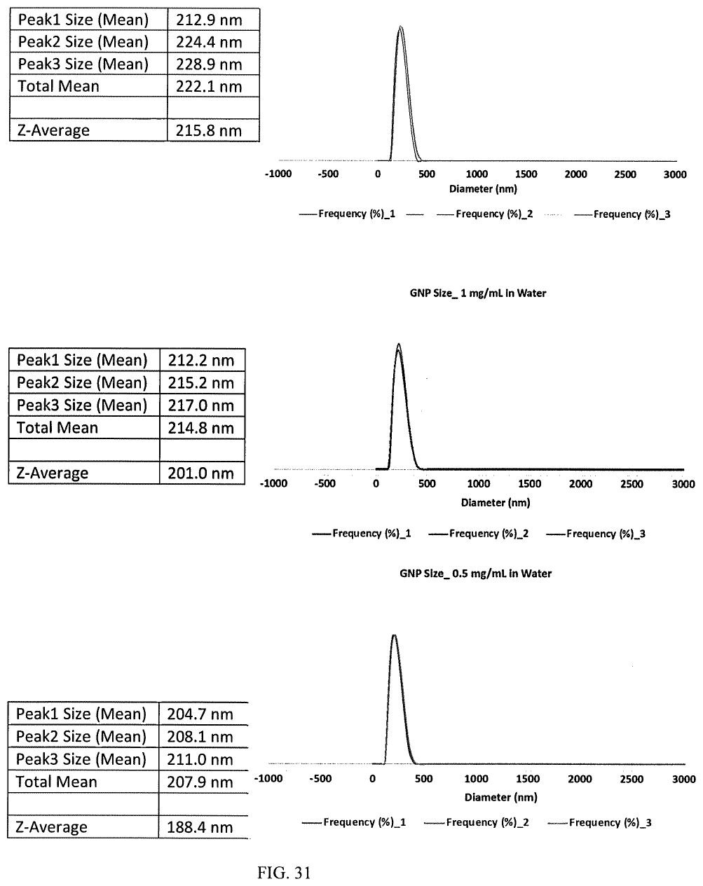

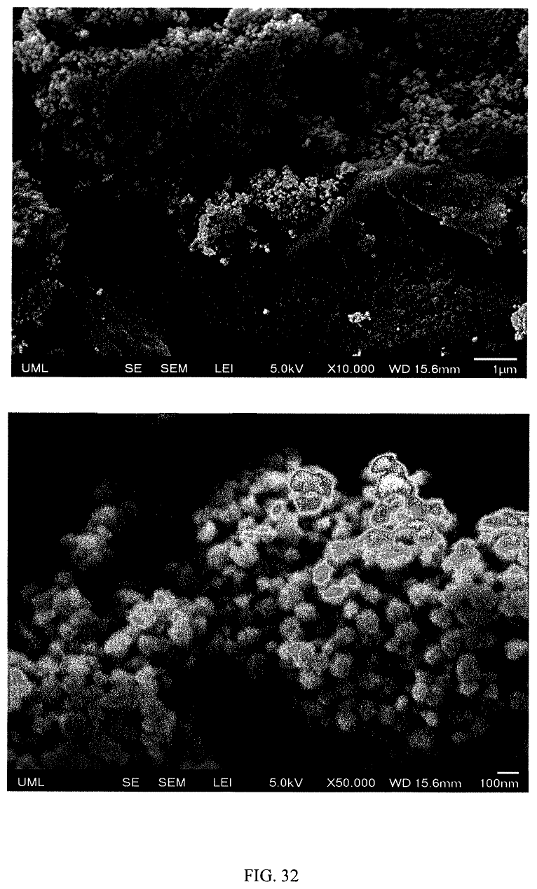

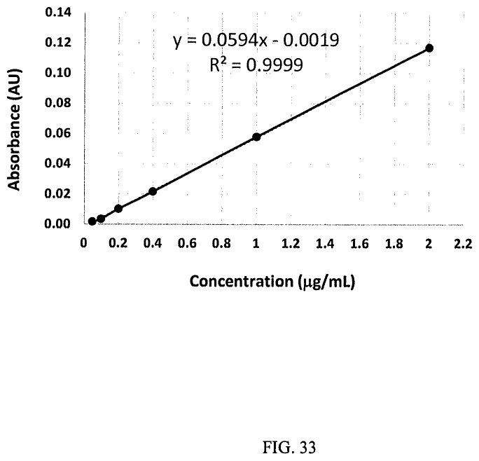

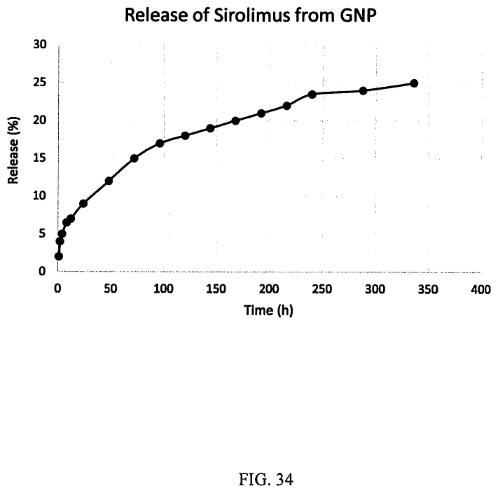

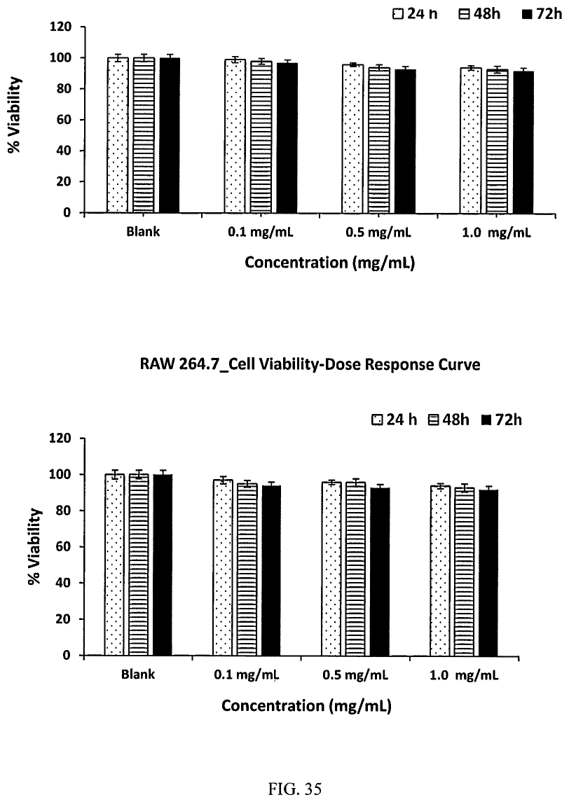

In certain embodiments, disruption of the target lesion and retraction of the device, are addressed. In addition, steps are taken to address the number of lumens profile needed of the system to accommodate functionality. In addition, steps are taken to limit the length of vessel to be treated, e.g., see FIG. 13. In one embodiment, a device comprises a hollow empty space (hard boundaries) created between two balloons, located longitudinally on top of one another with a space longitudinally located in between these 2 balloons where the space allows blood flow in between these balloons. Each balloon has multiple pores, e.g., micropores, along their luminal sides, and are filled with a drug solution, e.g., a Sirolimus solution, in either 100% DMSO or a mixture of DMSO and saline with little leakage through pores during travel through vessels to the treatment area. During deployment where the device is held in place, balloons are simultaneously inflated, just enough that the sides with the pores are tightly sticks to the vessel walls followed by inflation of the balloons to create pressure, e.g., squeezing, in order to allow the drug solution to pour out of the balloons through the pores and onto the vessel wall tissue. Absorption of DMSO into the vessel walls is contemplated as quick with a high delivery efficiency. The space between the two balloons is enough to allow continual blood throughflow which should ensure that the drug solution does not contact the blood stream and hence no loss of drug due to the flush of blood stream through this area as compared to losses using a conventional Drug Coated Balloon (DCB). Exemplary FIG. 11.

In one embodiment, a delivery system is provided comprising a medical grade flexible hollow plastic tube perforated with micropores around the outside of the tube, having end enclosures impermeable to fluids, wherein when filled with a fluid, e.g., a DMSO drug formulation, the fluid does not move through the micropores. Upon deployment in a treatment area, micropores would be adjacent to vessel walls and not the main stream of blood. Such plastic hollow tubes have outside boundaries of very thin and flexible plastic or textile (impermeable to fluid) which upon deployment in a vessel, responds to shear pressure generated by blood flow through the center area wherein the blood's hydrostatic pressure pushes the outer area of tubing against the vessel wall while pushing the drug formulation through the micropores and onto the vessel wall tissue. In other words, after the slowly moving blood through the narrow opening during deployment, when the tubes are held in position, blood then enters into the enlarging opening with greater force and higher volume as the tube is pushed towards the vessel walls for delivery an active agent, then tubes begin to deflate due to loss of fluid introduced into surrounding vessel wall tissue. Exemplary FIG. 12.

In one embodiment, a method of treatment using a device as described herein for treating thickened vessel wall areas, e.g., atherosclerotic plaques, PAD, etc., comprises preparation of the plaque covered vessel wall intended for treatment in the form of restoring blood flow through the plaque either using angioplasty or atherectomy. Thus, in one preferred embodiment, a method of treatment incorporates an angioplasty or atherectomy procedure prior to treatment with a device described herein, for combining within a complete procedure into a method of using the device. In one embodiment, atherectomy procedures include but are not limited to, rotational, transluminal, and directional atherectomy. In one embodiment, atherectomy procedures are used for below the knee applications.

There are at least two ways to stop the movement of emboli into the extremities that arise distal of devices used for treatment. One is filtration of the blood, in other words using a device having an attached filter or using a device a distal protection filter. Another is evacuating the disrupted tissue using a catheter so as not to allow distal movement of the emboli downstream of blood flow. Yet, another is by using a method for treating arthrosclerosis comprising a chemical method of dissolving or removing the atherosclerotic plaque combined with delivering a treatment to the vessel wall in contact with plaque by using a device as described herein.

In one embodiment, a device described herein having microholes in the outer surface provides a chemical profusion through the hypotube (infusion through the center of the hydrotube and irrigating into the vessel thought the microholes (micropores) depicted in FIG. 12. Infusion would have a period long enough for the chemical formulation to dissolve the target lesion but short enough so as to not cause clinical issues associated with stopping blood flow. Once sufficient erosion of the target lesion is achieved, the fragmented lesion would be evacuated through the evacuation catheter also depicted in FIG. 12. Once the lesion is removed, flow is sufficiently restored, and particulate/emboli is evacuated the drug solution could then be infused into the target area.

Aspects of this embodiment comprise a hypotube to keep a low profile of the delivery system also allowing moving through difficult lesions also known as “pushability”. An evacuation catheter with an open lumen located in an inner area between the two balloons so that blood and emboli can be removed, along with an associated proximal balloon for stopping flow. A restriction catheter telescoping over a hypotube comprising pores/holes, allows for variable length allowing expulsion of drug from open holes, but not from those holes covered by the surrounding catheter, provides a means of treatment to different lesion lengths using one device/system. See, exemplary FIG. 13.

Thus, in one embodiment, a means to change the length and/or distance the drug is being introduced into the vasculature is provided, therefore accommodating variations in lesion length. In this embodiment, treating varying lengths of a lesion is contemplated to be-accomplished in several ways. In some embodiments, the flow of solvent out of a solvent delivery channel, such as a hypotube (or other examples, such as a delivery catheter, conduit, channel, etc.) is changed-by including a sleeve/catheter or hypotube sliding over the solvent delivery channel for increasing or decreasing flow. Thus, in a preferred embodiment, the sleeve and solvent delivery channel move relative to one another. In other words, when holes are present in the solvent delivery channel, the sleeve (e.g., catheter) would prevent solvent from coming out of the side holes of the solvent delivery channel.

It is not intended that use of inventions described herein, be limited by the lesion length, indeed a variety of lengths can be treated. The table 1 below shows exemplary lesions lengths that may be treated. In one embodiment, lesions that may be treated range from 11-26 mm.

TABLE 1
Describing exemplary lesions lengths that may be treated.

| | Mean (SD), mm | | | | | | | |
|---|---|---|---|---|---|---|---|---|
| | Lesion Length | | | | Reference Diameter | | | |
| Study* | SES | | PES | | SES | | PES | |
| CORPAL,²³ 2005 | 26.0 | (14.0) | 24.0 | (14.0) | 2.96 | (0.40) | 2.94 | (0.40) |
| ISAR-DESIRE,²⁴ 2005 | 14.10 | (7.90) | 14.50 | (9.10) (9.5) | 2.61 | (0.47) | 2.66 | (0.48) |
| ISAR-DIABETES,²⁵ 2005 | 13.80 | (7.60) | 12.40 | (7.70) | 2.70 | (0.50) | 2.75 | (0.56) |
| REALITY,²⁶ 2005 | 16.96 | (10.04) | 17.31 | (10.09) | 2.40 | (0.48) | 2.40 | (0.48) |
| SIRTAX,²⁷ 2005 | 11.80 | (6.80) | 12.40 | (7.20) | 2.82 | (0.40) | 2.82 | (0.43) |
| TAXI,²⁸ 2005 | 16.0 | (2.0) | 14.0 | (2.0) | 3.20 | (0.10) | 3.20 | (0.10) |
| Overall | 16.77 | (9.65) | 16.45 | (9.74) | 2.69 | (0.43) | 2.69 | (0.43) |

Abbreviations: PES, paclitaxel-eluting stent; SES, sirolimus-eluting stent.
*Kastrati, et al., “Sirolimus-Eluting Stents vs Paclitaxel-Eluting Stents in Patients With Coronary Artery Disease Meta-analysis of Randomized Trials. Meta-analysis of Randomized Trials”.
JAMA, Vol 294, No. 7, 2005.
²³de Lezo, et al., “Drug-eluting stents for complex lesions: randomized rapamycin versus paclitaxel CORPAL study” [abstract]. J Am Coll Cardiol. 2005; 45(suppl A): 75A.
²⁴Kastrati, et al., “ISAR-DESIRE Study Investigators. Sirolimus-eluting stent or paclitaxel-eluting stent vs balloon angioplasty for prevention of recurrences in patients with coronary instent restenosis: a randomized controlled trial.” JAMA. 2005; 293: 165-171.
²⁵Kastrati, “Paclitaxel-eluting stent versus sirolimus eluting stent for the prevention of restenosis in diabetic patients with coronary artery disease (ISAR-DIABETES)”. Paper presented at: 2005 Scientific Session of the American College of Cardiology; Mar. 6, 2005; Orlando, Fla.
²⁶Morice, “Eight-month outcome of the REALITY study: a prospective, randomized, multi-center head-to-head comparison of the sirolimus-eluting stent (Cypher) and the paclitaxel-eluting stent (Taxus)”. Presented at: 2005 Scientific Session of the American College of Cardiology;
Mar. 6, 2005; Orlando, Fla.
²⁷Windecker, “Nine-month outcome of the SIRTAX trial: a randomized comparison of a sirolimus- with a paclitaxel-eluting stent for coronary revascularization.” Presented at: 2005 Scientific Session of the American College of Cardiology; Mar. 6, 2005; Orlando, Fla.
²⁸Goy, et al., “A prospective randomized comparison between paclitaxel and sirolimus stents in the real world of interventional cardiology: the TAXI trial.” J Am Coll Cardiol. 2005; 45: 308-311.

### Cationic Gelatin Nanoparticles (GNP) Delivery of Drug.

In some embodiments, drug loaded (e.g., drug encapsulated) nanoparticles are delivered within an artery. In some embodiments, drugs included but are no limited to drugs for treatment of vessel walls, such as plaque covered inside artery walls. In preferred embodiments, drug delivery is accomplished by using a device of the present inventions.

### Synthesis and Characterization Data of Prepared Gelatin Nanoparticles (GNP)

The following describes data obtained while preparing unloaded Gelatin Nanoparticles (GNP) and unloaded Cationic Gelatin Nanoparticles (GNP). Unloaded GNPs are prepared without a drug.

### 1. Preparation of Unloaded Cationic Gelatin Nanoparticles (GNP)

In one embodiment, a two-step desolvation method was used to prepare unloaded cationic gelatin nanoparticles.

Briefly, gelatin (2.5 g, type B; 300 g Bloom) was dissolved in ultrapure water (50 mL) in a 40° C. water bath while stirring at 600 rpm for 30 min. As described herein, Bloom refers to a strength of a gel or gelatin as a number of grams called the Bloom value. Most gelatins are between 30 and 300 g Bloom. The higher a Bloom value, the higher the melting and gelling points of a gel, and the shorter its gelling times. At this first desolvation step, acetone (50 mL) was added slowly to the gelatin solution and the mixture was allowed to stir at 600 rpm for another 30 min. After the precipitate or lump has settled at the bottom of the beaker, the clear supernatant (which contains lower molecular weight gelatin) was discarded. Under a constant stirring, the settled precipitated was dissolved in a fresh portion of ultrapure water (50 mL) and the pH was adjusted to 2.5-3 by the addition of 2 N hydrogen chloride (1.5 mL).

Then, a second desolvation step was carried out by adding acetone (140 mL) dropwise using a dropping funnel while magnetically stirring at 600 rpm followed by the dropwise addition of glutaraldehyde (12.6 mL, 50% concentration) solution in acetone (20 mL) for the stabilization of the nanoparticles as a crosslinking agent. Afterwards, the whole suspension was stirred at 600 rpm for 12 h (hour). Subsequently, the nanoparticles were centrifuged at 14000 rpm for 30 min (minutes) to collect nanoparticles. The pellet was redispersed in deionized (DI) water by sonicating at 30° C. for 30 min then centrifuged to remove glutaraldehyde and acetone. This purification process was repeated for at least two more times. Finally, the obtained GNP pellets were lyophilized overnight and collected as an off-white powder (2.0 g, 80% yield). The obtained cationic GNP were stored at 4° C. and were analyzed for its size of NPs. See, FIG. 31 and FIG. 32 for data on these cationic GNPs.

Non-limiting examples of nanoparticles include, poly(lactic-co-glycolic acid) (PLGA) nanoparticles, e.g., such as gelatin-poly (lactic-co-glycolic acid) nanoparticles, and gelatin nanoparticles (GNPS) as described herein.

### 2. Preparation of Sirolimus-Loaded Cationic GNPs

A drug loading study was carried out by incorporation (e.g. encapsulation) of Sirolimus simultaneously with nanoparticle production. In this study, Sirolimus:gelatin ratio was used as 1:35 in order to achieve high drug loading efficiency.

Briefly, gelatin (100 mg, type B; 300 g Bloom) was dissolved in ultrapure water (5 mL) in a 40° C. water bath while stirring at 600 rpm for 30 min. At the first desolvation step, acetone (5 mL) was added slowly to the gelatin solution and the mixture was allowed to stir at 600 rpm for another 30 min. After the precipitate or lump has settled at the bottom of the beaker, the clear supernatant (contain lower molecular weight gelatin) was discarded. Under a constant stirring, the settled precipitated was dissolved in a fresh portion of ultrapure water (5 mL) and the pH was adjusted to 2.5-3 by the addition of 2 N hydrogen chloride (150 μL). After that Sirolimus solution (300 μL) is added dropwise which is prepared by dissolving Sirolimus (10 mg) in DMSO (1 mL) at room temperature. Gelatin-drug solution was homogenized by stirring with a magnetic stirrer for 15 min.

Then, a second desolvation step was carried out by adding acetone (15 mL) dropwise using a glass pipette while magnetically stirring at 600 rpm followed by the dropwise addition of glutaraldehyde (120 μL, 50% concentration solution) for the stabilization of the nanoparticles as a crosslinking agent then the whole suspension was stirred at 600 rpm for 12 h. Subsequently, the nanoparticles were centrifuged at 14000 rpm for 30 min to collect nanoparticles. The supernatant was immediately subjected to UV-Vis Spectroscopy to evaluate the amount of the free Sirolimus that is not encapsulated. The settled pellet was redispersed in DI water by sonicating at 30° C. for 30 min and centrifuged to remove any glutaraldehyde and acetone. This purification process was repeated for two more times.

Finally, the obtained GNP pellets were lyophilized overnight and collected as an off-white powder (80 mg, 80% yield). The obtained Sirolimus encapsulated cationic GNP were stored at −20° C. and were analyzed for its encapsulation efficiency and capacity.

### 3. Sirolimus Encapsulation in GNP Study

To analyze the unknown amount of the Sirolimus we prepared calibration curve using UV-Vis Spectroscopy. The known amount of Sirolimus was dissolved in the mixture of water and ethanol (1:1 (v/v)) to prepare a stock solution with a final concentration of 1.0 mg/mL. The calibration curve ranged from 50 ng/mL-2 μg/mL. The absorbance peak at 288 nm corresponds to Sirolimus. A linear curve was obtained upon plotting concentration against the respective absorbance that generated R² value of 0.9999. The calibration curve was utilized to calculate the unknown amounts of Sirolimus. See, FIG. 31, Sirolimus calibration curve and encapsulation analysis.

### Entrapment Efficiency:

The amount of unloaded Sirolimus was quantified spectrophotometrically at 288 nm in the supernatant after the Sirolimus loaded GNPs were centrifuged at 14000 rpm. The drug entrapment efficiency (EE) was calculated according to the formula given below:

$$E E ( \% ) = ( \frac{Initial Sirolimus amount - Free Sirolimus amount}{Initial Sirolimus amount} ) \times 100$$

### Loading Efficiency

In a similar fashion loading efficiency can also be calculate using the following formula:

$$LE ( \% ) = ( \frac{Initial Sirolimus amount - Free Sirolimus amount}{Amount of GNP} ) \times 100$$

TABLE 2
Entrapment Efficiency (EE) and Loading
Efficiency (LE) for Sirolimus:GNP 1:35.

| Drug formulation | | |
|---|---|---|
| Sirolimus:GNP | EE (%) | LE (%) |
| 1:35 | 99.18 | 2.98 |

Entrapment Efficiency (EE) indicates the percent of drug that has been encapsulated and, in this case, it is more that 99%. This means the formulation was highly efficient in encapsulating Sirolimus. Whereas. The LE signifies the percent of Sirolimus trapped by the given amount of nanoparticles. In this experiment LE was found to be 2.98% meaning that 2.98 mg is trapped in every 100 mg of GNP.

### 4. Kinetic Release of Sirolimus Over Two Weeks

The release of sirolimus from the GNP was monitored in phosphate-buffered saline (PBS; pH 7.4) at 37° C. under gentle shaking. In brief, 1 ml of GNP nanoparticles (1 mg/ml) was transferred to a dialysis bag (10-kDa cutoff; Sigma-Aldrich) and immersed into 10 ml of release buffer. At predetermined time intervals, 0.5 ml of release buffer was removed for measurement, and the same amount of fresh buffer was added back. The solution was measured for the presence of the sirolimus using a UV-Vis spectroscopy at 280 nm. The concentration of the sirolimus present in the removed buffer was determined using the calibration curve shown in FIG. 33. The release kinetics shows a sustained release (25% of the initial dose) of the sirolimus over period of 2 weeks. See, FIG. 34, Release kinetics of sirolimus from cationic GNP under sink conditions (i.e. related to the dissolution testing procedure).

### 5. Cell Viability Analysis

THP-1 and RAW 264.7 cell lines were used in order to assess the cytotoxic potential of empty-cationic GNP. Cells were maintained in DMEM with stable glutamine including 10% fetal bovine serum (FBS) and 1× penicillin streptomycin mixture. Cells were grown at 37° C., in a 5% CO₂ humidified incubator. The effect of empty GNP on cell viability was analyzed by MTT ([3-(4,5-dimethylthiazol-2-yl)-2,5-diphenyltetrazolium bromide]assay). Firstly THP-1 and RAW 264.7 cells were seeded in 96-well plates (1×10⁴ cells/well). After 24 h incubation, the cells were treated with empty GNPs. Untreated cells served as control. At the end of certain incubation times (24 h, 48 h, and 72 h), the medium was replaced with 100 μL fresh growth medium and then 10 μL MTT solution (5 mg/mL) was added. After 4 h incubation, the medium with MTT in the wells was discharged carefully and 100 μL of DMSO were added to solubilize formazan crystals formed by the reactions in living cells. Absorbances were measured at 540 nm with UV spectrophotometer. See, FIG. 35, Cell Viability test results.

### EXPERIMENTAL

The following Example 1 describes and evaluates Sirolimus treatment of isolated porcine arteries.

### Example 1

### Materials and Methods

### 1. Exemplary Reagents and Materials, Sources.

Sirolimus/Rapamycin (>98%, AdooQ Bioscience), Ascomycin (>98%, AdooQ Bioscience), (see FIG. 26), dimethyl sulfoxide (DMSO, 99.9%, Sigma Aldrich). Methanol (>99.9%, Sigma Adrich), and ammonium acetate (>99.9%, Sigma Aldrich) HPLC grade. All other reagents were of analytical grade and were used without any further purification.

### 2. Instrumentation and Analytical Conditions

Sirolimus/Rapamycin was analyzed using an LC-MS/MS system consisting of an Agilent 1290 (San Jose, CA, USA) coupled to electrospray ionization on a triple quadrupole mass spectrometer (Agilent 6460, San Jose, CA, USA) equipped with a turbo ion spray interface in negative ionization mode, and an Agilent LC 1200 Binary pump system (Agilent Technologies, Santa Clara, CA, USA). Ascomycin was used as an internal standard (IS). A Gemini 5 μm NX-C18 110 Å, LC Column polar-RP 80A column (50×2 mm, Phenomenex, Torrance, CA, USA) equipped with a Security Guard™ column (4.0 mm×3.0 mm) was utilized to optimally separate sirolimus and the internal standard from endogenous substances of porcine carotid artery. The mobile phase was a mixture of 20 mM ammonium acetate (A) and methanol (B). Sample separation was conducted using a flow rate of 0.2 mL/min and a 10-min gradient condition (0-1 min: 20% B, 1-6 min: 100% B, 6-7 min: 100% B, 7.1-10 min: 20% B). The injection volume was L for calibration and carotid artery samples. The selected reaction monitoring transitions of m/z 912.5→371.2 and m/z 790.5→530.3 were applied for sirolimus and IS, respectively. Mass data were acquired using Analyst software version 1.5.2 (Applied Biosystems-SCIEX, Concord, ON, Canada). Data analysis was processed using SCIEX OS offline software version 1.6 (Applied Biosystems-SCIEX, Concord, ON, Canada). The working parameters of the LC-MS/MS system are listed in Table 3.

TABLE 3
LC-MS/MS working parameters.

| Source Parameters | Sirolimus | Ascomycin (IS) |
|---|---|---|
| Gas Temperature (° C.) | 350 | 350 |
| Capillary potential (V) | 3500 | 3500 |
| Collision energy (V) | 37 | 33 |
| Gas flow (L/min) | 12 | 12 |
| Sheath gas (° C.) | 400 | 400 |
| Nozzle voltage (V) | 500 | 500 |

### 3. Solutions and Validation Samples

Sirolimus/Rapamycin was dissolved in acetonitrile to prepare a stock solution (1 mg/mL) and including IS with 2 ng/μL and were gradually diluted by the serial dilution method using calibrated pipettes (2-20 μL, 10-100 μL and 100-1000 μL), in order to obtain working stock dilutions at decreasing concentrations (1, 2.5, 5, 10, 25, 50, 100, 250, 500 and 1000 μg/μL). These solutions were used for the preparation of mass spectrometry optimization, calibration curve and quality control standards in DMSO and carotid artery homogenates. These stock solutions and stock dilutions were stored at −20° C., respectively. Exemplary calibration data is shown in FIGS. 27A, 27B and 27C.

### 4. Rapamycin Delivery in Carotid Artery

Sirolimus/Rapamycin was dissolved in DMSO to prepare a final concentration of 100 mg/mL (stock solution) and stored at −80° C. The porcine carotid arteries were washed 5 times in PBS (1×) to remove all the blood and fluids, followed by cutting them into 40 mm length. The surface area (SA) of the artery was calculated to be 628 mm² (lateral SA_{cylinder}=2πrh; π=3.14, r=2.5 mm, and h=40 mm). The Rapamycin solution was diluted in DMSO to prepare 1256 g/mL (2 μg/mm²). One of the carotid arteries was tied at one end and then it was filled with 0.5 mL of Rapamycin solution ([Rapamycin]₀=628 μg/mL) then the open end was tied. After a 5-minute treatment, the solution was drained out, then arteries were washed with 5×0.2 mL of PBS (1×) which was also collected for the analysis. A similar experiment was performed as a biological duplicate. The arteries and the washings were then analyzed for the presence of Sirolimus/Rapamycin for determining the amount that was absorbed by the tissues compared to the amount that was washed out for use as a model.

### 5. Samples Preparation: Tissue Homogenization

Treated and untreated porcine carotid artery segments (100 mg, 2 mm) were shredded in methanol with a surgical blade and transferred to a 2 ml tube with a total volume of 1 ml methanol and 100 μL of internal standard (1 ng/ml). This tube contained garnet beads/shards for homogenizing tissue. Then a Qiagen Tissue Lyser LT was used to beat the artery shreds at 50 Hz for 10 minutes. The supernatant was dried down completely under a gentle stream of nitrogen using a nitrogen evaporator. The samples were resuspended in 100 μL of DMSO and placed in inserted universal autosampler vials prior to analysis. The concentrations of rapamycin from the artery and washings were found to be 19.28 g/mL (30.13% of total drug in 2 mm of artery) and 3.14 g/mL (0.5% of total drug in 40 mm of artery).

### Results:

TABLE 4
Rapamycin concentrations.

| Total Dose | 220 ug |
|---|---|
| Lost in Blood stream (washed out) | 1 ug |
| Remaining in tissue | 68.75 ug OR 30.13% of the dose |
| | (remaining drug was recovered) |
| Delivery efficiency | 99.55% |

### Example 2

Data Summary from Animal Experiments

The purpose of this non-Good Laboratory Practice (GLP) study is to test mitotic drugs, Paclitaxel and Sirolimus, that penetrate vascular tissue. A swine model was recruited for an in-vivo study to evaluate the penetration of the drug formulation and their resident time in the vessels over 2 weeks. The study consists of three female Yorkshire swine (50-60 Kg) that survived for 1, 7, and 14 days.

For each survival timepoint, one animal was euthanized, and the drug present in the arteries and local toxicity were examined by High-performance liquid chromatography (HPLC) and histology. Baseline and endpoint whole blood were collected for each animal in a tube containing an anticoagulant, such as citrate or heparin.

Formulations: Two Different Formulations were Prepared:
1. Paclitaxel was dissolved in pure medical grade DMSO to achieve a final concentration of 1 mg/mL. 15 mg of paclitaxel was dissolved in 15 ml of DMSO. Each test site was injected with 0.5 mL of this formulation that constitutes of 500 mg of paclitaxel.
2. Sirolimus was encapsulated in gelatin nanoparticles (GNP). By calculation, 100 mg of GNP contained 3 mg of sirolimus. 100 mg of GNP were dispersed in 15 mL of DMSO in saline (1:1 (v/v)) When injected the final concentration of the sirolimus was 160 mg.

There were 8 test sites per animal (4. Paclitaxel, and 4. Sirolimus) and all test sites were performed by accessing both left and right femoral arteries. The animals were sedated and prepared in a sterile operating room (OR) and the animal placed in a dorsal recumbency position.

An incision of 5-8 cm was made alongside the femoral (or carotid) artery, and surrounding muscle and perivascular fascia were bluntly dissected to expose the artery.

Once the artery is exposed, the following is an exemplary method for drug testing:
The test sites were marked, approximately 10-20 mm (4 on each side).
Both ends were occluded of the test site to stop the blood flow.
The blood from the test site was evacuated using a catheter.
The test site was filled with the drug solution designated for that site (e.g., 0.5 mL).
The solution was held in the test site for a 5-minute exposure.
After 5 minutes the drug formulation was evacuated.
The normal blood flow was restored.

The above steps were repeated for each of the 8 sites per animal (4 sites per side). The right side of the animal was injected with Paclitaxel formulation and the other side (left) with Sirolimus.

### Observations:

1. All the animals were found to be healthy on the day of necropsy. However, the first animal for t=24 h was found to have an adverse reaction of severe edema on the right leg around surgery (paclitaxel injected). When the surgeon cut open the sutures, a lot of dead tissues and blood clots around the site of delivery was observed. This occurrence of edema and blood clots around the delivery site could either be due to surgery or the effect of pure DMSO and high doses of paclitaxel.

Whereas the left leg that was injected with nanoparticles encapsulated with Sirolimus dispersed in 50% DMSO in saline was found to have not caused any edema and nor blood clots near the delivery site. This lack of adverse reaction suggests that surgery did not cause edema or blood clots. However, an adverse reaction might be due to the presence of paclitaxel and DMSO.
2. However, an adverse reaction was not observed in animals on day 7. This animal was healthy, and no edema was observed. Same lack of reaction was observed in the day 14 animal.
3. Blood sample was collected before necropsy and save at −80° C. For each day, 8 femoral arteries (4 from each leg) were harvested and two of each of 1 cm length was saved in HPLC grade methanol at −80° C. until drug isolation and other 2 from each was saved for histology. Along with the arteries, a 0.5 cm muscle tissue around the testing site of right leg was harvested for the HPLC analysis to evaluate paclitaxel amounts.

### Results:

The HPLC facility isolated the paclitaxel in usual manner and sirolimus was isolated with an addition step of enzymatic digesting of the GNP with trypsin at 37° C. before the analysis of each of them by Liquid Chromatography with tandem mass spectrometry (LC-MS-MS).
1. Paclitaxel on day 1 in both the arteries with the final concentration of 12 ng/g in the first artery and 5.2 ng/g in second artery was detected by LC-MS/MS. The solution that was evacuated after 5 min of the formulation in the test site was evaluated by LC-MS/MS to detect 15% (75 mg) of the initial concentration of injected paclitaxel solution. This suggests that 85% (425 mg) of the paclitaxel was absorbed by the tissue. Only few nanograms were detected in 24 h and that could indicate that probably 100% DMSO as a carrier took away the paclitaxel from the site to other region of the leg muscles that were not evaluated.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, cell biology, molecular biology, biochemistry, chemistry, or related fields are intended to be within the scope of the following claims.

1-49. (canceled)

50. A method of treating a target tissue exhibiting neointimal growth in a vessel in a subject, comprising:
a) occluding said vessel in said subject with a device, at least a portion of said device positioned downstream past the target tissue exhibiting neointimal growth, said portion stopping blood flow at the distal end of said vessel;
b) introducing a treatment solution comprising rapamycin and dimethylsulfoxide (DMSO) so that it contacts said target tissue for a period of time, wherein said rapamycin is encapsulated in a plurality of nanoparticles; and
c) removing said device from said vessel, thereby returning blood flow at the distal end of said vessel.

51. The method of claim 50, wherein said device is a hypotube comprising a channel, said channel having one or more openings.

52. The method of claim 51, wherein said treatment solution is introduced at step b) through said channel of said hypotube and through said one or more openings.

53. The method of claim 51, wherein said hypotube comprises a filter.

54. The method of claim 53, wherein said filter is deployed like an umbrella at the distal end to stop blood flow at said distal end.

55. The method of claim 51, wherein said portion of said device positioned downstream is an inflatable balloon associated with said hypotube.

56. The method of claim 50, wherein said vessel contains a partial or complete blockage at or near said target tissue.

57. The method of claim 56, further comprising, prior to step b), performing a procedure to remove or reduce said blockage.

58. The method of claim 57, wherein said procedure is a chemical procedure that dissolves at least a portion of said blockage.

59. The method of claim 58, wherein said chemical procedure comprises delivering a chemical through said channel of said hypotube for a period of time, said hypotube further comprising microholes such that the blockage is irrigated by the chemical coming through the microholes.

60. The method of claim 57, wherein said procedure is a surgical procedure.

61. The method of claim 60, wherein said surgical procedure is angioplasty.

62. The method of claim 60, wherein said surgical procedure is an atherectomy.

63. The method of claim 62, wherein said atherectomy is selected from the group consisting of rotational, transluminal, and directional atherectomy.

64. The method of claim 57, further comprising, prior to step b), but after performing said procedure to remove or reduce said blockage, removing particles generated by said procedure.

65. The method of claim 64, wherein said particles are potential emboli and they are removed with an evacuation catheter.

66. The method of claim 50, wherein said vessel is a popliteal vessel.

67. The method of claim 50, wherein said target tissue exhibiting neointimal growth is in a vessel below the knee of said subject.

68. The method of claim 50, wherein said device has an associated proximal balloon which is positioned upstream to block proximal blood flow.

69. The method of claim 50, wherein said DMSO is diluted in saline.

70. The method of claim 69, wherein said DMSO is diluted in saline as a 50% solution.

71. The method of claim 69, wherein said DMSO is diluted in saline as a 20% solution.

72. The method of claim 50, wherein said rapamycin is encapsulated in gelatin nanoparticles.

73. A method of treating a target tissue exhibiting neointimal growth in a vessel in a subject, comprising:
a) providing a device comprising a hypotube comprising a channel and one or more openings, said hypotube having an associated distal balloon;
b) introducing said device into said vessel in said subject, at least a portion of said device positioned downstream past the target tissue exhibiting neointimal growth, said portion comprising the associated distal balloon;
c) inflating said associated distal balloon thereby stopping blood flow at the distal end of said vessel;
d) introducing a treatment solution comprising a growth inhibitor into said hypotube and out of said one or more openings so that said treatment solution contacts said target tissue for a period of time, wherein said treatment solution comprises rapamycin is encapsulated in a plurality of nanoparticles; and
e) deflating said associated distal balloon and removing said device from said vessel, thereby returning blood flow at the distal end of said vessel.

74. The method of claim 73, wherein said treatment solution comprises rapamycin and dimethylsulfoxide (DMSO).

75. The method of claim 74, wherein said DMSO is diluted in saline.

76. The method of claim 74, wherein said DMSO is diluted in saline as a 50% solution.

77. The method of claim 74, wherein said DMSO is diluted in saline as a 20% solution.

78. The method of claim 73, wherein said rapamycin is encapsulated in gelatin nanoparticles.

79. The method of claim 73, wherein said vessel contains a partial or complete blockage at or near said target tissue.

80. The method of claim 79, further comprising, prior to step d), performing a procedure to remove or reduce said blockage.

81. The method of claim 80, wherein said procedure is a chemical procedure comprises delivering a chemical solution that dissolves at least a portion of said blockage, said chemical solution being different from said treatment solution.

82. The method of claim 81, wherein said chemical solution is delivered through said channel of said hypotube and out of said one or more openings such that the blockage is irrigated by the chemical solution for a period of time.

83. The method of claim 80, wherein said procedure is a surgical procedure.

84. The method of claim 83, wherein said surgical procedure is angioplasty.

85. The method of claim 83, wherein said surgical procedure is an atherectomy.

86. The method of claim 85, wherein said atherectomy is selected from the group consisting of rotational, transluminal, and directional atherectomy.

87. The method of claim 79, further comprising, prior to step d), but after performing said procedure to remove or reduce said blockage, removing particles generated by said procedure.

88. The method of claim 87, wherein said particles are potential emboli and they are removed with an evacuation catheter.

89. The method of claim 73, wherein said vessel is a popliteal vessel.

90. The method of claim 73, wherein said target tissue exhibiting neointimal growth is in a vessel below the knee of said subject.

91. The method of claim 73, wherein said device further comprises an associated proximal balloon which is positioned upstream of the target tissue

92. The method of claim 91, further comprising, after step b) inflating said associated proximal balloon to block proximal blood flow.

93. A system, comprising i) a hypotube comprising a channel, said channel having one or more openings, said hypotube having an associated distal balloon and an associated proximal balloon, ii) an evacuation catheter with an open lumen to the inner section between the distal and proximal balloons; iii) a restriction catheter to allow for variable length to accommodate different lesion lengths; and iv) a treatment solution comprising rapamycin encapsulated in a plurality of nanoparticles dispersed in DMSO diluted with saline.

94. The system of claim 85, wherein the restriction catheter is configured to cover the hypotube delivering the nanoparticles.

95. The system of claim 86, wherein the restriction catheter can be moved distally approximately in relation to the distal balloon to restrict the amount of therapeutic and location of the therapeutic being eluted by the hypotube, thereby allowing for the variable length of the vessels and lesions therein.

96. A delivery system comprising a delivery device comprising first, second and third ports, said first port configured to supply fluid through a first channel, said fluid for inflating an angioplasty balloon, said second port configured to supply fluid through a second channel, said fluid for inflating a distal balloon to block blood flow, said third port configured to supply fluid through a third channel, for supplying a treatment solution into a vessel, wherein said treatment solution comprises rapamycin encapsulated into a nano carrier dispersed in DMSO diluted with saline.

97. The method of claim 96, wherein said first channel is within a first hypotube, said second channel is within a second hypotube, and said third channel is within a third hypotube.
